(19) United States
(12) Patent Application Publication
(10) Pub. No.: US 2024/0207216 A1
(43) Pub. Date: Jun. 27, 2024

(54) COMPOSITIONS AND METHOD FOR TREATING GUT PERMEABILITY CAUSED BY HYDROGEN SULFIDE PRODUCING BACTERIA

(71) Applicants: UNM RAINFOREST INNOVATIONS, Albuquerque, NM (US); The United States Government as represented by the Department of Veterans Affairs, Washington, DC (US)

(72) Inventors: Henry C. Lin, Albuquerque, NM (US); Sudha B. Singh, Albuquerque, NM (US); Cristina N. Coffman, Albuquerque, NM (US)

(21) Appl. No.: 18/390,242

(22) Filed: Dec. 20, 2023

Related U.S. Application Data
(60) Provisional application No. 63/433,817, filed on Dec. 20, 2022.

(51) Int. Cl. A61K 31/29; A61K 33/06; A61K 33/24

(52) U.S. Cl. CPC A61K 31/29; A61K 33/06; A61K 33/24

(57) ABSTRACT

A method of treating a subject having, or at risk of having, leaky gut caused by sulfate reducing bacteria generally includes administering to the subject an amount of a Snail inhibitor or an inhibitor of sulfate reducing bacteria effective to reduce ameliorate at least one symptom or clinical sign of leaky gut caused by sulfate reducing bacteria. A method of inhibiting deterioration of cellular junctions in cells contacted with sulfate reducing bacteria generally includes contacting the cells with an amount of Snail inhibitor or an inhibitor of sulfate reducing bacteria effective to reduce the extent to which the sulfate reducing bacteria deteriorate cellular junctions compared to untreated cells.

### CROSS-REFERENCE TO RELATED APPLICATION

This application claims the benefit of U.S. Provisional Patent Application No. 63/433,817, filed Dec. 20, 2022, which are incorporated herein by references in their 10 entireties.

### SUMMARY

This disclosure describes, in one aspect, a method of treating a subject having, or at risk of having, leaky gut caused by hydrogen sulfide producing bacteria including. Generally, the method includes administering to the subject a composition that includes an amount of a Snail inhibitor or an inhibitor of hydrogen sulfide producing bacteria effective to reduce ameliorate at least one symptom or clinical sign of leaky gut caused by sulfate reducing bacteria.

In one or more embodiments, the composition is administered in an amount effective to inhibit overgrowth or bloom of hydrogen sulfide producing bacteria.

In one or more embodiments, the composition is administered in an amount effective to decrease Snail expression in treated cells compared to untreated cells.

In one or more embodiments, the composition is administered in an amount effective to decrease FITC-dextran flux in treated cells compared to untreated cells.

In one or more embodiments, the composition is administered in an amount effective to 25 decrease the percentage of cells with Snail localized in the nucleus in treated cells compared to untreated cells.

In one or more embodiments, the composition is administered in an amount effective to decrease cytoplasmic localization of occludin in treated cells compared to untreated cells.

In one or more embodiments, the composition is administered in an amount effective to 30 increase paracellular localization of occluding in treated cells compared to untreated cells.

In one or more embodiments, the hydrogen sulfide producing bacteria include sulfate reducing bacteria.

In another aspect, this disclosure describes a method of inhibiting deterioration of cellular junctions in cells contacted with hydrogen sulfide producing bacteria. Generally, the method includes contacting the cells with a composition that includes an amount of Snail inhibitor or an inhibitor of hydrogen sulfide producing bacteria effective to reduce the extent to which the hydrogen sulfide producing bacteria deteriorate cellular junctions compared to untreated cells.

In one or more embodiments, the composition is administered in an amount effective to decrease Snail expression in treated cells compared to untreated cells.

In one or more embodiments, the composition is administered in is an amount effective to decrease FITC-dextran flux in treated cells compared to untreated cells.

In one or more embodiments, the composition is administered in an amount effective to decrease the percentage of cells with Snail localized in the nucleus in treated cells compared to untreated cells.

In one or more embodiments, the composition is administered in an amount effective to decrease cytoplasmic localization of occludin in treated cells compared to untreated cells.

In one or more embodiments, the composition is administered in an amount effective to increase paracellular localization of occluding in treated cells compared to untreated cells.

In one or more embodiments, the hydrogen sulfide producing bacteria include sulfate reducing bacteria.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1. Desulfovibrio vulgaris (DSV) decreased transepithelial electrical resistance (TEER) and increased paracellular permeability in polarized Caco-2 cells. Polarized Caco-2 cells grown in 12-well 0.4 μm transwell inserts were treated with D. vulgaris with various MOIs for 24 hours. (A) TEER was measured using EVOM ohmmeter by inserting electrodes into the trans-wells. (B) Cells were incubated for one hour with 40 μl 4 kDa FITC-dextran (25 mg/ml) added to the apical surface. After one hour, 100 μl of the medium from basolateral side was removed and analyzed for FITC fluorescence, using excitation at 486 nm and emission at 528 nm. (C) FITC-dextran flux was monitored in cells infected with L. reuteri, B.fragilis, or D. vulgaris for 24 hours with different MOIs. (D) FITC-dextran flux in cells were infected with live bacteria, heat killed (HK) bacteria, bacterial Postgate's culture medium alone, or with filtered culture supernatant of live D. vulgaris bacteria. Graph in all panels represent Mean±SEM from at least three independent experiments. *p<0.05, ***p<0.0001.

FIG. 2. D. vulgaris induced changes in the localization of occludin. Polarized Caco-2 cells were infected with D. vulgaris (MOI20) for 24 hours. Cells were then fixed with 4% paraformaldehyde. After blocking in solution containing 5% FBS, 0.3% TritonX-100 in PBS, cells were incubated with anti-occludin antibody (in 1% BSA with 0.1% TritonX-100 in PBS) overnight at 4° C. Cells were washed with PBS and incubated further with secondary anti-rabbit Alexa Flour 488 antibody for two hours at room temperature. Cells were washed and mounted (PROLONG GOLD, Thermo Fisher Scientific, Inc., Waltham, MA). Cells were visualized by confocal microscopy (FLUOVIEW FV1200, Olympus Corp., Tokyo, Japan). Z-stacks were performed for each image and the final images were collected after compressing the z-stacks together.

FIG. 3. D. vulgaris (DSV) induced Snail protein expression. Polarized cells were treated with D. vulgaris (MOI 20) for 24 hours. (A) Snail detection by Western blot in Control and DSV-treated cells. Actin was used as a loading control. (B) Graph represents Mean±SEM from at least three independent experiments. *p<0.05. (C) Immunofluorescence of cells infected with or without D. vulgaris. Cells were fixed with 4% paraformaldehyde and stained with anti-Snail antibody followed by secondary antibody labeled with Alexa Flour 488. Cells were visualized by confocal microscopy (FLUOVIEW FV1200, Olympus Corp., Tokyo, Japan). Scale bar=20 μm.

FIG. 4. D. vulgaris induced permeability in Snail-dependent fashion. Polarized Caco-2 cells were transfected with either scrambled control (Scr) siRNA or siRNA against Snail (siSnail) for 48 hours. (A) transfection efficiency was determined by Western blotting in cells transfected with two siRNAs against Snail alone or in combination. (B) Transfected cells were infected with DSV for further 24 hours. After 24 hours, FITC-Dextran was added on the apical side of the cells for one hour following which 100 μl of medium from the basolateral surface was collected and analyzed for FITC-flux. (C) Snail expression was monitored by Western blotting in transfected cells challenged with D. vulgaris. (D) Graph represents Mean±SEM from at least 3 independent experiments. *p<0.05.

FIG. 5. Intestinal Alkaline Phosphatase inhibits DSV-induced permeability by inhibiting Snail. Polarized Caco-2 cells were pre-treated with IAP (500 U/ml) at the apical and the basolateral surface for 24 hours prior to challenge with D. vulgaris. (A) FITC-dextran was added to the cells apically after 24 hours infection with D. vulgaris. Fluorescence was measured by taking out 100 μl medium from the basolateral surface after one-hour incubation. Graph represents Mean±SEM of % FITC-flux compared to uninfected control cells from at least three independent experiments. **p<0.01. (B) Snail protein expression was analyzed by Western blotting in cells pre-treated with or without IAP followed by infection with D. vulgaris. The solution in which IAP was supplied was used as a Vehicle control. Actin was used as a loading control. (C) Quantification of Western blot for detection of Snail. Graph represents Mean±SEM of ratio of Snail/Actin and values were normalized to control from at least three independent experiments. *p<0.05. (D) Cells were pre-treated with or without IAP followed by infection with D. vulgaris. Cells were fixed with 4% paraformaldehyde and probed by immunofluorescence for Snail staining. Graph represents quantification of % cells showing nuclear staining of Snail in control, DSV+Vehicle (Veh), or DSV+IAP treated groups. Values represent Mean±SEM from three independent experiments. *p<0.05. (E) Cells were pre-treated with or without IAP followed by infection with D. vulgaris. Cells were fixed with 4% paraformaldehyde and probed by immunofluorescence for occludin staining as described above. Images were collected as Z-stacks that were compressed at the end to generate the final images. Scale bar=20 μm.

FIG. 6. Cells were pre-treated with scrambled (Scr) or siSnail followed by infection with D. vulgaris (DSV+Scr, DSV+si Snail). Control was treated with scrambled (Control+Scr). Cells were fixed with 4% paraformaldehyde and probed by immunofluorescence for occludin staining as described above. Images were collected as Z-stacks that were compressed at the end to generate the final images. Scale bar=20 μm.

FIG. 7. Rapamycin reduces DSV-induced paracellular permeability. Human Caco-2 cells were grown in 0.4 μm trans-well inserts for three weeks to generate polarized intestinal epithelial monolayers. Cells were treated with 100 nM rapamycin, an inducer of autophagy, for two hours followed by DSV infection (MOI 20) for 24 hours. Barrier integrity was assessed by measuring paracellular flux of 4 kDa FITC-Dextran.

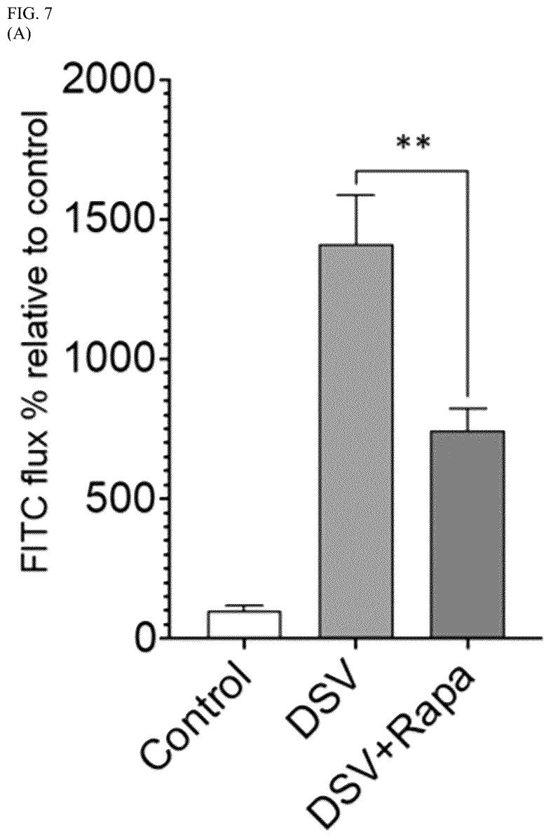

FIG. 8. DSV induces activation of PI3K/AktnTNF pathway in a time-dependent manner. (A) RAW macrophages (8×10⁵) were infected with DSV at MOI 20 for 5 minutes, 15 minutes, 30 minutes, 120 minutes, or 240 minutes. Cells were lysed and protein lysate was prepared. Fifty μg of protein lysate was separated on SDS-PAGE and analyzed for the expression of p-Akt, Akt, TNF-α, iNOS, actin, p-IκB, total IκB, p-NF-κB p65, and total p65 NF-κB by Western blotting. Actin was used as a loading control.

FIG. 9. Quantification of Western blots. Blots from FIG. 8 were quantified by analyzing the ratio of protein of interest/Actin. (A) p-Akt (S437)/Akt. (B) p-Akt (T308)/Akt. (C) p-IκB/IκB. (D) p-NF-κB/NF-κB. (E) TNF-α. (F) iNOS. For phosphorylated proteins, the ratio of phosphorylated/total protein was calculated. Data represents Mean±SEM from at least three independent experiments. Values were normalized to control. One-way ANOVA was used to determine the statistical significance. Values were compared to control, with a post-hoc Dunnett's test. *p<0.05, **p<0.01, ***p<0.001.

FIG. 10. Live and heat-killed DSV, but not the bacterial culture supernatant induces PI3k/Akt/TNF/iNOS pathway. (A) RAW cells were treated with Live or heat killed (HK) DSV (MOI 20) or bacterial culture supernatant (Sup) for four hours and harvested. Cells were lysed and protein lysate was prepared. Fifty μg of protein lysate was separated on SDS-PAGE and analyzed for p-Akt, AKt, TNF-α, and iNOS by Western blotting. Actin was used as a loading control. (B) Quantification of p-Akt-Akt Western blot. (C) Quantification of TNF-α Western blot. (D) Quantification of iNOS Western blot. Blots were quantified by analyzing the ratio of protein of interest/Actin. For phosphorylated Akt, the ratio of p-Akt/total Akt was calculated. Data represents Mean±SEM from at least three independent experiments. Values were normalized to control. One-way ANOVA was used to determine the statistical significance. Values were compared to Live DSV. *p<0.05.

FIG. 11. Comparison of effects of DSV and Lactobacillus plantarum on PI3k/Akt/TNF/iNOS pathway. (A) RAW cells were treated with DSV or Lacto (MOI 20) for four hours and harvested. Cells were lysed and protein lysate was prepared. Fifty μg of protein lysate was separated on SDS-PAGE and analyzed for p-Akt, Akt, TNF, and iNOS by Western blotting. Actin was used as a loading control. (B) Quantification of p-Akt-Akt Western blot. (C) Quantification of TNF-α Western blot. (D) Quantification of iNOS Western blot. Blots were quantified by analyzing the ratio of protein of interest/Actin. For phosphorylated Akt, the ratio of p-Akt/total Akt was calculated. Data represents Mean±SEM from at least three independent experiments. Values were normalized and compared to Control. One-way ANOVA was used to determine the statistical significance. *p<0.05, ***p<0.001.

FIG. 12. LY294002 inhibits DSV-induced activation of PI3K/Akt/TNF pathway. (A) RAW cells were treated with LY at either 10 μM or 50 μM for one hour before infection with DSV for four hours. Cells were lysed and protein lysate was prepared. Fifty μg of protein lysate was separated on SDS-PAGE and analyzed for p-Akt, Akt, TNF, iNOS, p-p70S6K, and total p70S6K by Western blotting. Actin was used as a loading control. (B) Quantification of p-Akt-Akt Western blot. (C) Quantification of p-P70/P70 Western blot. (D) Quantification of TNF-α/actin Western blot. (E) Post-infection, RAW cell culture supernatant was collected and proteins were precipitated using ethanol precipitation. Proteins were resuspended in PBS and 50 μg of proteins were loaded on SDS-PAGE and secreted TNF-α was detected using TNF-α antibody. Actin was used as a loading control.

FIG. 13. LY294002 inhibits DSV-induced activation of PI3K/Akt/TNF pathway. (A) Quantification of secreted TNF-α. (B) Cells were treated with LY at either 10 μM or 50 μM for one hour before infection with DSV for four hours. Protein lysate was analyzed for iNOS expression. (C) Cells were pretreated with or without LY 50 μM followed by infection with DSV for 30 minutes and protein samples were analyzed for p-p65 NF-κB and total NF-κB. (D) Blots were quantified by analyzing the ratio of p-NF-κB/NF-κB. Values were normalized and compared to control. Data represents Mean±SEM from at least three independent experiments. Values were normalized to control. One-way ANOVA was used to determine the statistical significance. Values were compared to DSV, with a post-hoc Dunnett's test. *p<0.05, **p<0.01, ***p<0.001.

FIG. 14. Activation of PI3/Akt pathway by DSV is dependent on TLR2 signaling. RAW cells were treated with TLR2 signaling inhibitor C29 at 200 μM for three hours before infection with DSV for four hours. Cells were lysed and protein lysate prepared. Fifty μg of protein lysate was separated on SDS-PAGE and analyzed for p-Akt, Akt, TNF, and iNOS by Western blotting. Actin was used as a loading control.

FIG. 15. Activation of PI3/Akt pathway by DSV is dependent on TLR2 signaling. (A) Quantification of p-Akt/Akt Western blot. (B) Quantification of p-P70/P70 Western blot. (C) Quantification of TNF-α Western blot. (D) Western blot of secreted TNF-α. (E) Quantification of TNF-α Western blot in (D). (F) Quantification of iNOS Western blot. Blots were quantified by analyzing the ratio of protein of interest/Actin. For phosphorylated proteins, the ratio of phosphorylated/total protein was calculated. Data represents Mean±SEM from at least three independent experiments. Values were normalized to control. A two-tailed t-test was used to compare DSV and DSV+C29. *p<0.05, **p<0.01.

FIG. 16. Rapamycin reduces DSV-induced Snail protein expression. Polarized Caco-2 cells were treated with 100 nM rapamycin for two hours, followed by DSV infection (MOI 20) for 24 hours. Cells were harvested and lysed. (A) Protein lysate was analyzed for Snail expression by Western blotting. (B) Quantitation of Snail expression from Western blot in (A). DSV: 7.14±0.73; DSV+Rapamycin: 3.10±0.74, **p<0.01.

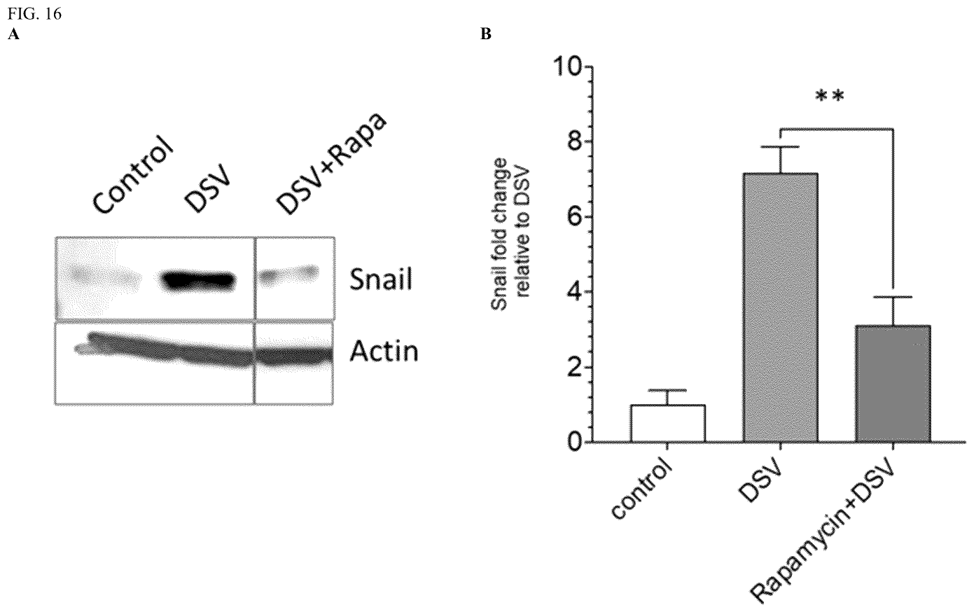

FIG. 17. Rapamycin reduced DSV-induced Snail mRNA expression. Caco2 cells were treated with 100 nM rapamycin for two hours followed by DSV infection (MOI 20) for 24 hours. RNA was isolated from samples and cDNA was synthesized from RNA. Quantitative PCR was performed for analyzing gene expression of Snail using specific TAQMAN probes (Roche Molecular Systems, Inc., Pleasanton, CA).

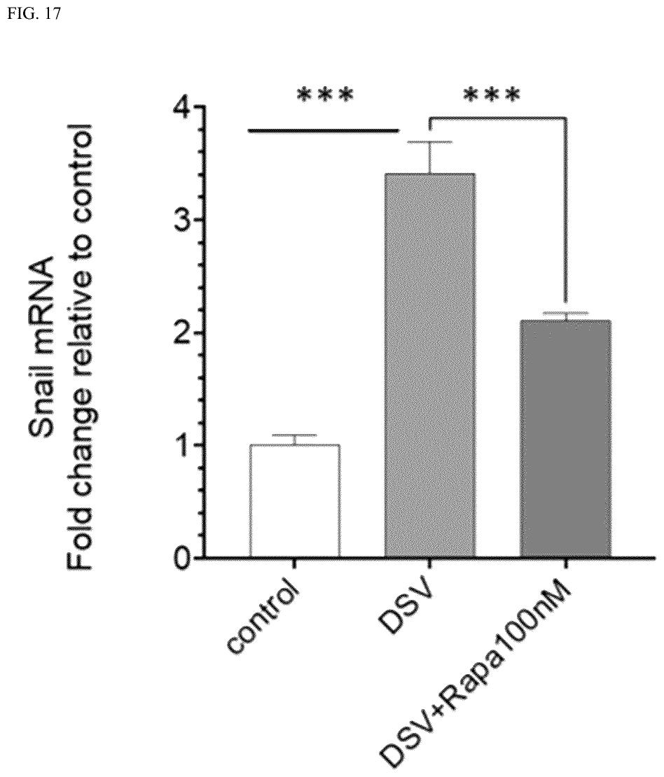

FIG. 18. Rapamycin reduces DSV-induced Snail nuclear translocation. Caco-2 cells were treated with 100 nM for two hours, followed by DSV infection (MOI 20) for 24 hours. Cells were fixed with 4% paraformaldehyde and blocked with a solution of 5% FBS and 0.3% Triton-X100 for one hour. This was followed by incubation of both apical and basolateral surface of the trans-wells with primary antibody against Snail1 in 1% BSA and 0.3% TritonX-100. Cells were then washed with PBS followed by incubation with secondary anti-rabbit antibody. Imaging was done with a confocal microscope.

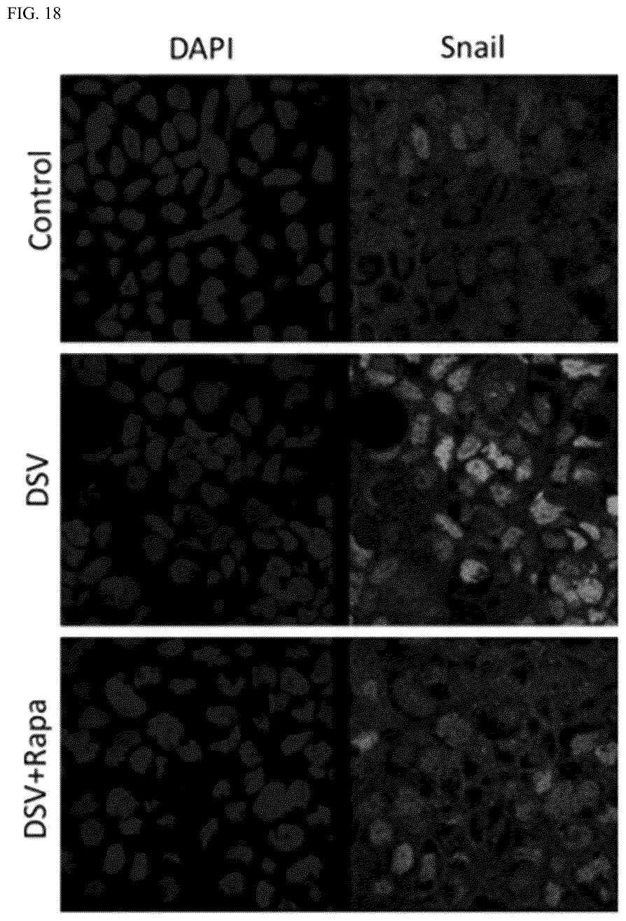

FIG. 19. Schematic diagram illustrating exemplary modes of action of inhibitors of DSV-induced Snail expression.

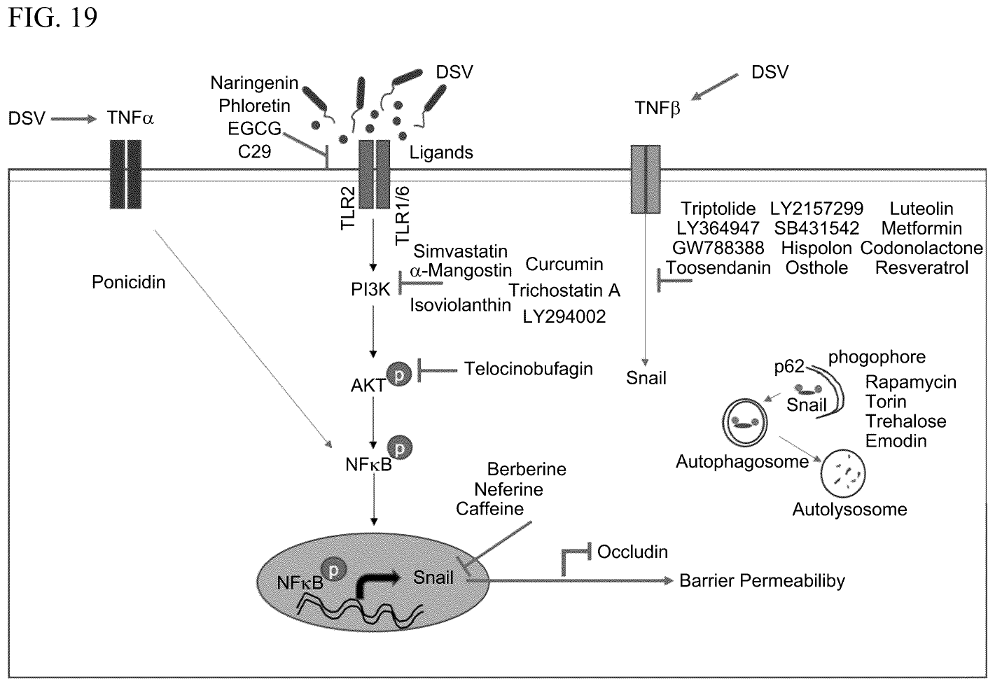

FIG. 20. Small intestine Fusobacterium nucleatum density in antibiotic-treated mice. Quantitative PCR of ileum from control and antibiotic-treated mice (ABX). Data are presented as Mean±SEM. *P<0.05.

### DETAILED DESCRIPTION OF ILLUSTRATIVE EMBODIMENTS

This disclosure describes methods for treating increased gut permeability caused by hydrogen sulfide producing bacteria including but not limited to sulfate reducing bacteria. In some embodiments, the methods generally involve administering a Snail inhibitor or an inhibitor of sulfate reducing bacteria or another hydrogen sulfide producing bacteria to a subject having, or at risk of having, leaky gut.

Intestinal epithelium is a formidable barrier that protects the host against luminal bacteria, harmful molecules, and pathogens. Barrier integrity is mainly dependent on tight junctions (TJs) that include proteins such as claudins, occludin, and zonula occludens; adheren junctions (AJs) that include cadherins and catenins; desmosomes that include desmogleins and desmocollins; and gap junctions. Together, these junctions minimize paracellular flux and microbial translocation. Dysfunction of barrier integrity with increased intestinal permeability (leaky gut) has been linked to many diseases associated with gut microbial dysbiosis such as IBS, IBD, Parkinson's diseases, and metabolic syndrome.

Sulfate reducing bacteria (SRB) are residents of the gut found mainly in the colon in humans and in animals. SRB uses sulfate as terminal electron acceptor and produces hydrogen sulfide through its reduction. As used herein, SRB is therefore used generically to refers to a microorganism that reduces any sulfur compounds. SRB are usually minor members of the gut but are often found in increased numbers (bloom) in several conditions associated with leaky gut such as inflammatory bowel disease (IBD). Many of these conditions associated with leaky gut are associated with an abnormal expansion of the gut microbiome from the colon to the small intestine or small intestinal bacterial overgrowth (SIBO). Among SRB, Desulfovibrio is a prominent genus. Desulfovibrio spp. increase in number in various intestinal and extraintestinal diseases such as IBD, pouchitis, periodontitis, and Parkinson's disease. Not all hydrogen sulfide-producing bacteria generate this gas through reduction of inorganic sulfur molecules. There are microbes that use sulfur containing amino acids. Among these bacteria are members of the genera Fusobacterium. Overgrowth of Fusobacterium is observed in diseases such as inflammatory bowel disease and colorectal cancer and may contribute to disease development.

While described herein in the context of exemplary embodiments in which D. vulgaris and Fusobacterium nucleatum are used as model hydrogen sulfide producing bacteria, the methods described herein may be practiced using any hydrogen sulfide-producing bacteria including, but not limited to, sulfate reducing bacteria. Exemplary sulfate reducing bacteria include members of the genus Desulfovibrio, members of the genus Desulfobacter, members of the genus Desulfomonas, members of the genus Desulfobulbus, and members of the genus Desulfotomaculum, members of the genus Desulfomicrobium, members of the genus Desulfohalobium, members of the genus Desulfonatronum, members of the genus Desulfoarculus, members of the genus Desulfobotulus, members of the genus Desulfulonema, members of the genus Desulfobacterium, members of the genus Desulfomonile, members of the genus Desulfococcus, members of the genus Desulfobacula, members of the genus Desulfofustis, members of the genus Desulfospira, members of the genus Desulforhopalus, members of the genus Desulfocella, members of the genus Desulfobacca, members of the genus Desulfocapsa, members of the genus Desulfovibrio, members of the genus Desulfosacina, members of the genus Desulfosporosinus. Additional exemplary hydrogen sulfide-producing bacteria include, but are not limited to, member of the genus Fusobacterium (e.g., F. nucleatum, F. necrophorum, F. ulcerans, F. varium, F. necrogenes, F. perfoetans, F. gonidiaformans, or F. russi), members of the genus Prevotella, members of the genus Leptotrichia, members of the genus Veillonella, members of the genus Atopobium, selected members of the genus Clostridium, selected members of the genus Escherichia, selected members of the genus Salmonella, selected members of the genus Klebsiella, selected members of the genus Enterobacter, selected members of the genus Bilophila, selected members of the genus Bacteroides, selected members of the genus Peptostreptococcus, selected members of the genus Odoribacter, selected members of the genus Pyramidobacter, selected members of the genus Flavonifractor, selected members of the genus Porphomonas, selected members of the genus Roseburia, or selected members of the genus Streptococcus.

Snail (sometimes referred to as Snail) is a transcription factor responsible for increased intestinal permeability by negatively regulating tight junction proteins such as occludin, claudins, and ZO-1, and adheren junction proteins such as E-cadherin. siRNA against Snail inhibits tight junction permeability induced by various experimental stimuli. Snail expression is upregulated by pathogens such as Salmonella, Group B Streptococcus, and H. pylori, but the effect of resident commensal bacteria such as sulfate reducing bacteria on Snail is not known.

In one aspect, this disclosure describes the use of Snail inhibitors and/or inhibitors of sulfate reducing bacteria to treat intestinal permeability caused by sulfate reducing bacteria. Intestinal alkaline phosphatase (IAP) is an exemplary Snail inhibitor. IAP is a host defense protein produced by enterocytes in the small intestine and is secreted into the intestinal lumen, blood, and stool. Several protective functions have been attributed to IAP including lipopolysaccharide (LPS) detoxification, regulation of bicarbonate secretion in the small intestine, regulation of gut microbiome, dephosphorylation of proinflammatory nucleotides, and induction of anti-inflammatory autophagy.

While exemplified herein in the context of exemplary embodiments in which the inhibitor of Snail expression in is intestinal alkaline phosphatase (IAP), the compositions and methods described herein can include the use of any inhibitor of Snail expression, whether the inhibition is the result of direct inhibition of Snail expression or the result of inhibiting regulators of Snail that otherwise would induce Snail expression. This disclosure identified inhibitors of Snail expression that directly downregulate Snail production, its nuclear translocation, and/or Snail and function. This disclosure also identifies inhibitors of Snail expression that target pathways that act upstream of Snail and that induce Snail expression and activation. Exemplary positive regulators of Snail that are targets of inhibition include, but are not limited to, TLR-2 pathway, PI3K/Akt pathway, NF-κB, TGF-β, TNF-α, and Pak1.

This disclosure also identifies pathways that directly inhibit Snail expression including, but not limited to, GSK3p and autophagy. Phosphorylation of Snail by GSK3p at various serine positions (e.g., S¹⁰⁷, S¹¹¹, S¹¹⁵, S¹¹⁹), causes nuclear export of Snail and its and degradation by proteasomes, thus inhibiting Snail function. Autophagy is a well-known eukaryotic intracellular degradative pathway that has many direct and indirect roles in protecting the gut. Genome wide association studies identified polymorphisms in autophagy genes such as IRGM and ATG16L1 as susceptibility loci in the occurrence of Inflammatory Bowel Diseases (IBD). Known functions of autophagy in the intestine include, but are not limited to, its direct antimicrobial function, its regulation of antimicrobial proteins such as lysozyme, its ant-inflammatory role, and regulating gut barrier integrity by controlling the tight junction proteins and mucus that make up the formidable physical barrier.

Autophagy inhibits Snail transcription factor. Snail is known for its causal role in epithelial-mesenchymal transition (EMT) that is central to metastasis in cancer progression. As described above, Snail also is responsible for increasing intestinal permeability by inhibiting tight junction protein expression and assembly. Autophagy degrades Snail in various cellular models while autophagy inhibition causes an increase in Snail expression. Thus, the suppressive effect of autophagy on Snail may also help maintain the barrier integrity by inhibiting Snail-mediated inhibition of tight junction protein function. Some of the inducers of autophagy that may aid in maintaining intestinal barrier integrity include, but are not limited to, starvation, rapamycin, resveratrol, intestinal alkaline phosphatase (IAP), zinc, and Vitamin D.

While described herein in the context of using intestinal alkaline phosphatase as a model inhibitor of Snail expression, the methods described herein can involve the use of any inhibitor of Snail expression. Exemplary inhibitors of Snail expression include, but are not limited to, an inhibitor of TLR2 signaling, an inhibitor of PI3K/Akt signaling, an inhibitor of TGF-β signaling, an inhibitor of TNF-α, an inhibitor of NF-κB, an inhibitor of Pak1, an inducer of GSK3p, or an inducer of autophagy. Thus, exemplary inhibitors of Snail expression include, but are not limited to, ponicidin, naringenin, phloretin, epigallocatechin-3-gallate (EGCG), C29, simvastatin, a-mangostin, curcumin, isoviolanthin, trichostatin A, LY294002, telocinobufagin, an F-box ligase (e.g., β-TrCP1/Fbxw1, FBXL14, Fbxl5, FBX011, Fbxo45, FBW7, etc.), SOCS box protein SPSB3, HECT domain E3 ubiquitin ligase 1 (HECTD1), LKB1/STK11, palbociclib (by targeting CDK4/6), thiolutin (by inhibiting deubiquitinase PSMD14), CYD19 (by disrupting interaction of Snail with CBP/p300), GSK3p (drives ubiquitination and degradation via β-TrCP1), toosendanin, luteolin, berberine, metformin, caffeine, neferine, hispolon, osthole, codonolactone, triptolide, galunisertib (LY2157299), LY364947, SB431542, GW788388, resveratrol, rapamycin, torin, emodin, or trehalose. Also, the methods described herein can involve the use of an inhibitor of sulfate reducing bacteria. Exemplary inhibitors of sulfate reducing bacteria include, but are not limited to, bismuth subsalicylate, molybdate, or magnesium.

### Sulfate Reducing Bacteria Decreased TEER and Increased Paracellular Permeability in Polarized Caco-2 Cells

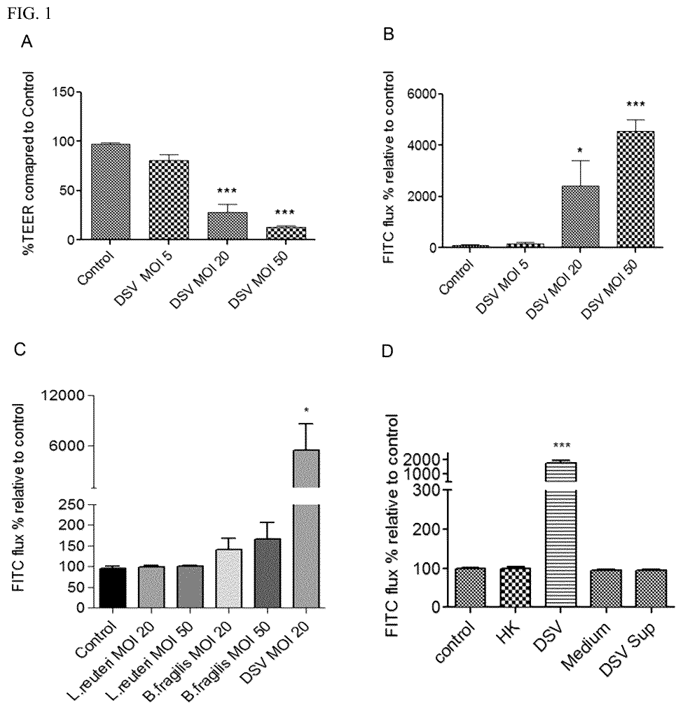

D. vulgaris induced increased intestinal permeability in Caco-2 cells as measured by reduced transepithelial electrical resistance (TEER) and/or increased FITC-dextran flux. Polarized Caco-2 cells were treated for 24 hours with D. vulgaris using different multiplicities of infection (MOI) (FIG. 1). D. vulgaris challenge caused a significant reduction in TEER at MOI 20 and at MOI 50 compared to control uninfected cells (FIG. 1A). Next, paracellular permeability was measured by monitoring the flux of 4 kDa FITC-dextran through the trans-well. MOI of 20 and MOI of 50 caused a significant increase in FITC-flux compared to control cells (FIG. 1B). As MOI 20 was the lowest dose of D. vulgaris that caused significant increase in permeability, this dose was used for all subsequent experiments.

The effect of bacteria belonging to the most predominant phyla in the gut—i.e., with gram negative Bacteroidetes (B.fragilis) and gram positive Firmicutes (L. reuteri)—on Caco-2 permeability were compared to D. vulgaris by measuring FITC-dextran flux. In contrast to D. vulgaris causing a significant increase in permeability (FIG. 1C), L. reuteri or B.fragilis did not, even at a MOI 50 (FIG. 1C). Thus, induction of increased intestinal permeability is not a universal feature of commensal bacteria in the gut and may be caused by selected opportunistic bacteria or pathobionts such as D. vulgaris or other sulfate reducing bacteria that are found to bloom in various diseases.

Next, heat-killed (HK) D. vulgaris were added to Caco-2 cells (comparable to MOI 20 of live bacteria) for 24 hours to test whether the increase in permeability was the property of live D. vulgaris or whether it could also be induced by dead bacteria. Further, D. vulgaris culture supernatant was added to Caco-2 cells to determine whether the increase in permeability was a property of live D. vulgaris or the property of a secretory factor. Live bacteria were pelleted and the supernatant was passed through a 0.2-μm filter to remove bacteria. Cells were incubated with either the filtered supernatant (DSV Sup) or bacterial growth medium alone as a control (Medium) for 24 hours. Only live D. vulgaris increased the FITC-flux compared to the control (FIG. 1D). Neither the heat-killed bacteria nor the culture supernatant increased FITC-flux (FIG. 1D) had this effect, suggesting that induction of increased intestinal permeability could only be caused by live sulfate reducing bacteria and not triggered by a structural property of the bacteria or a bacterial secretory product.

Sulfate reducing bacteria induce changes in the localization of TJ protein occludin

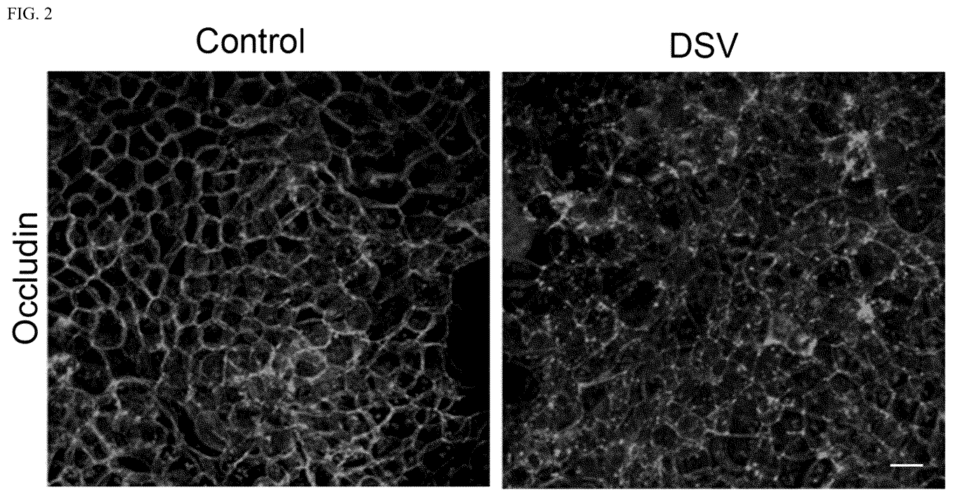

The mechanism by which sulfate reducing bacteria induced increased gut permeability was investigated by analyzing the protein expression of tight junction proteins such as occludin and claudin-2. Western blot analysis did not show any downregulation of any of these proteins in response to D. vulgaris. Next, permeability changes were analyzed by immunofluorescence. While D. vulgaris did not inhibit protein expression of occludin, it caused a remarkable shift in the localization of this protein (FIG. 2) from a distinct and sharp staining of paracellular spaces to a loss of this localization with disorganized displacement to the cytoplasm. Thus, loss of discrete localization of occludin from the paracellular space explains the increased permeability that represents impaired barrier function.

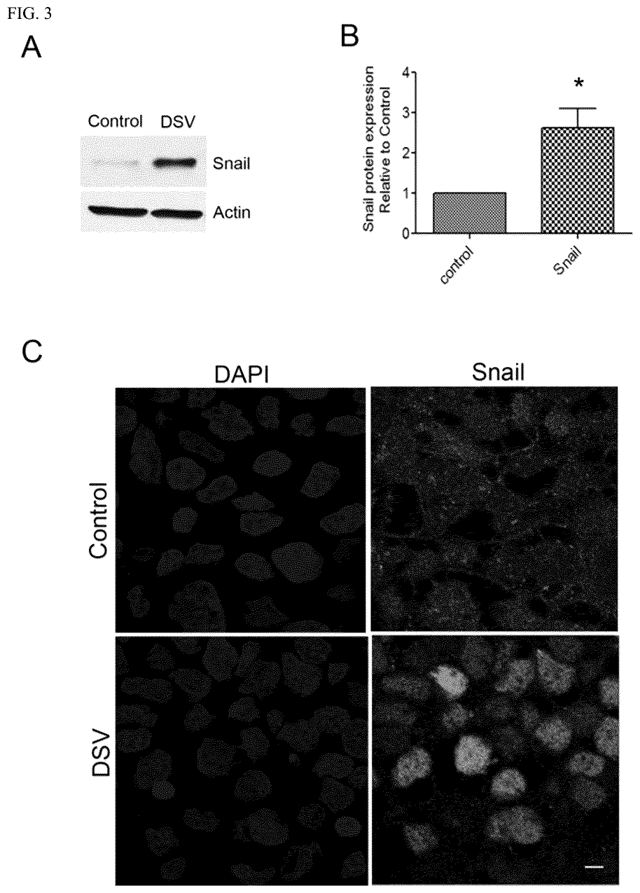

Sulfate reducing bacteria induce Snail protein expression Snail is a transcription factor that disrupts barrier integrity by downregulating the expression of tight junction proteins such as occludin. Snail also downregulates expression of genes for adheren junction proteins such as E-cadherin. D. vulgaris induces Snail protein expression in Caco-2 cells. Compared to control cells, D. vulgaris-treated cells had a significant upregulation of Snail protein expression (FIG. 3A, 3B). Next, the nuclear translocation of Snail in cells infected with D. vulgaris was analyzed. All control cells displayed a diffuse cytoplasmic Snail staining (FIG. 3C). After D. vulgaris infection, however, nuclear staining of Snail could be observed in approximately 50% cells. Thus, sulfate reducing bacteria induced Snail protein expression and promoted its nuclear translocation.

### Sulfate Reducing Bacteria Induce Barrier Permeability in a Snail-Dependent Fashion

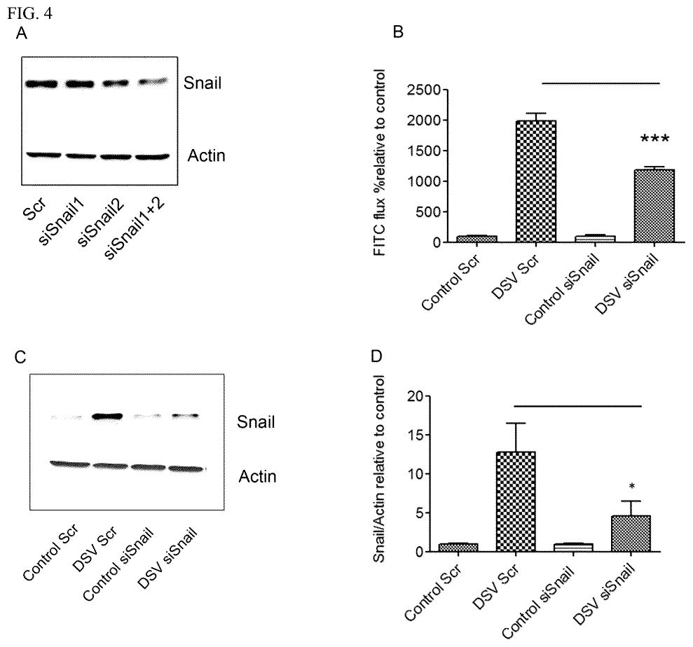

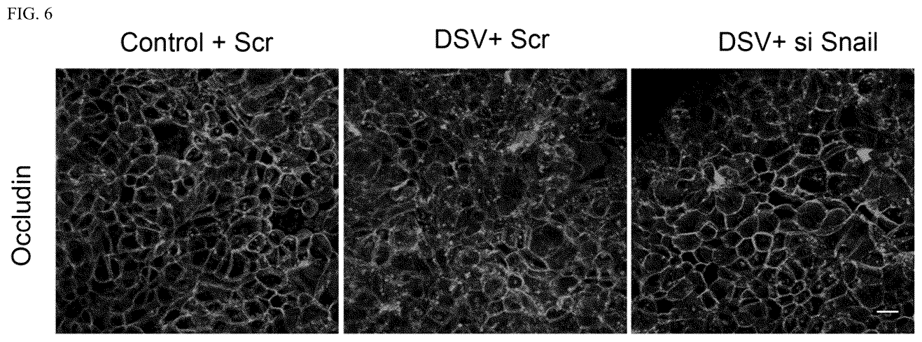

Next, the dependence of D. vulgaris-induced permeability on Snail expression was tested. For this, cells were first transfected with either scrambled siRNA (Scr) as control or with siRNA against Snail. Forty-eight hours after transfection, cells were infected with D. vulgaris (FIG. 4). To get the optimal transfection efficiency, two siRNAs against Snail were tested independently and in combination (FIG. 4A). The best efficiency was obtained in the combined siRNA group. Based on these results, the combination of two siRNAs to knock down Snail expression was used in subsequent experiments (referred to as siSnail). D. vulgaris induced a marked increase in FITC-dextran flux in cells transfected with Scr siRNA (FIG. 4B). However, a significant decrease in FITC-dextran flux was observed after D. vulgaris infection in cells transfected with Snail siRNA compared to Scr-transfected cells. Similarly, a significant reduction in Snail expression in response to D. vulgaris was observed in siSnail cells compared to D. vulgaris-infected Scr cells (FIG. 4C, 4D). Finally, the extent to which inhibiting Snail expression could reduce D. vulgaris-induced disruption of occludin localization was tested. Cells had an obvious change in the staining of occludin from uniform cellular junction (Control+Scr) to a disorganized cytoplasmic staining (DSV+Scr) (FIG. 6). However, in cells transfected with Snail siRNA (DSV+si Snail), the disruption of D. vulgaris-induced paracellular staining of occludin was reduced. Taken together, these results suggest that barrier permeability induced by sulfate reducing bacteria occurs in a Snail-dependent manner.

Snail Inhibitor Intestinal Alkaline Phosphatase Reversed D. vulgaris-Induced Permeability

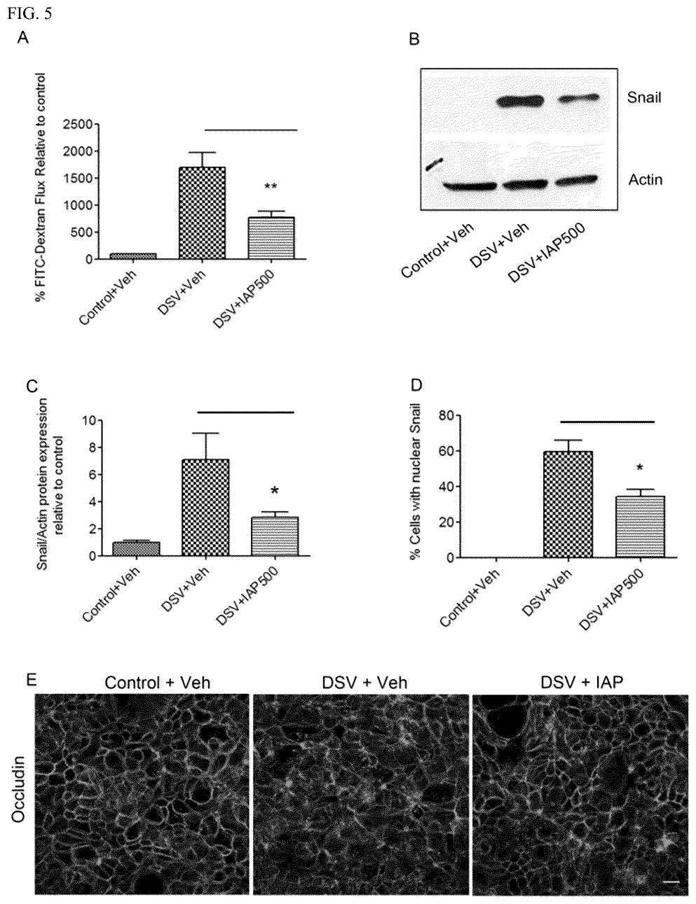

Intestinal alkaline phosphatase (IAP) is a host defense protein secreted by the small intestinal cells that affects the intestinal barrier via its effect on tight junction proteins such as occludin. The extent to which IAP, as a model inhibitor of Snail, reduced permeability induced by the model sulfate reducing bacterium D. vulgaris was tested. Cells were pre-treated with IAP or with vehicle alone for 24 hours before infection with D. vulgaris (FIG. 5A). Treatment of cells with IAP inhibited D. vulgaris-induced FITC-dextran flux compared to DSV+Veh treatment (FIG. 5A). Next, induction of Snail protein expression by D. vulgaris in the presence of IAP was tested. Snail expression was significantly reduced in cells pre-treated with IAP compared to DSV+Veh-treated cells (FIG. 5B, 5C).

Further, there was a significant reduction in the percentage of cells with nuclear localization of Snail in DSV+IAP treated cells compared to DSV+Veh-treated cells (FIG. 5D). Control cells had no nuclear Snail staining. These results suggest that IAP inhibited D. vulgaris-induced Snail expression and its nuclear localization.

Finally, immunolocalization of occludin in D. vulgaris-treated cells was examined in the absence or presence of IAP. In DSV+Veh cells, there was a disruption in the cellular junction staining of occludin to a more cytoplasmic and diffuse staining pattern compared to Control+Veh cells. However, in the presence of IAP (DSV+IAP), occludin staining appeared comparable to control (FIG. 5E).

Together, these data suggest that a Snail inhibitor (e.g., intestinal alkaline phosphatase) inhibited increased permeability by inhibiting Snail.

Thus, the model sulfate reducing bacterium Desulfovibrio vulgaris (DSV) increased barrier permeability in polarized Caco-2 cells. D. vulgaris serves as a model sulfate reducing bacterium since it belongs to the most abundant genus, Desulfovibrio, among sulfate reducing bacteria that overgrow or bloom in conditions associated with gut microbial dysbiosis. Thus, while described herein in the context of an exemplary embodiment in which the hydrogen sulfide producing bacteria are sulfate reducing bacteria as exemplified by D. vulgaris, the compositions, methods, and treatments described herein can involve reducing leaky gut induced by any hydrogen sulfide producing bacteria in the gut. Exemplary hydrogen sulfide producing bacteria include, but are not limited to, gram negative mesophilic sulfate reducing bacteria, and/or gram-positive spore-forming sulfate reducing bacteria. All use sulfate as a terminal electron receptor during anaerobic respiration. Two families of gram-negative mesophilic sulfate reducing bacteria are found within the delta subdivision of the Proteobacteria, the Desulfovibrionaceae and the Desulfobacteriaceae. The genera Desulfovibrio, Desulfomicrobium, Desulfohalobium, and Desulfonatronum are included in Desulfovibrionaceae. The genera Desulfoarculus, Desulfobulbus, Desulfobotulus, Desulfobacter, Desulfulonema, Desulfobacterium, Desulfomonile, Desulfococcus, Desulfobacula, Desulfofustis, Desulfospira, Desulforhopalus, Desulfocella, Desulfobacca, Desulfocapsa, Desulfovibrio, and Desulfosacina. Gram-positive spore forming sulfate reducing bacteria include the genera Desulfotomaculum and Desulfosporosinus. Additional exemplary hydrogen sulfide-producing bacteria include, but are not limited to, member of the genus Fusobacterium (e.g., F. nucleatum, F. necrophorum, F. ulcerans, F. varium, F. necrogenes, F. perfoetans, F. gonidiaformans, or F. russi), members of the genus Prevotella, members of the genus Leptotrichia, members of the genus Veillonella, members of the genus Atopobium, selected members of the genus Clostridium, selected members of the genus Escherichia, selected members of the genus Salmonella, selected members of the genus Klebsiella, selected members of the genus Enterobacter, selected members of the genus Bilophila, selected members of the genus Bacteroides, selected members of the genus Peptostreptococcus, selected members of the genus Odoribacter, selected members of the genus Pyramidobacter, selected members of the genus Flavonifractor, selected members of the genus Porphomonas, selected members of the genus Roseburia, or selected members of the genus Streptococcus.

This disclosure describes the effects of hydrogen sulfide producing bacteria on barrier permeability and the mechanism by which the model sulfate reducing bacterium D. vulgaris (DSV) serves as an example of bacterial influence on barrier permeability. Sulfate reducing bacteria induce the expression and nuclear translocation of the transcription factor Snail. In addition, this disclosure describes treating cells with a model Snail inhibitor (the host defense protein intestinal alkaline phosphatase) inhibited DSV-induced barrier dysfunction by inhibiting Snail expression that is induced by D. vulgaris.

Depending upon their classification, resident commensal bacteria may affect barrier integrity differently. Probiotics such as Lactobacillus spp. can enhance the tight junction barrier. Similarly, bacteria such as F. prausnitzii, R. intestinalis, and B. faecalis can improve barrier function. In contrast, pathogens such as Salmonella and opportunistic commensals such as E. coli K12 induce intestinal permeability. Among sulfate reducing bacteria, F. nucleatum, a proinflammatory bacterium associated with various diseases such as periodontitis, cancer, and colitis, induces barrier permeability in vivo and in vitro. F. nucleatum also inhibited the expression of ZO-1 and occludin in the colon and affected their distribution. In Caco-2 cells, F. nucleatum induced permeability as assessed by increased FITC-dextran flux as well as by a loss of TEER. The data presented in this disclosure relating to D. vulgaris-induced permeability extend these findings. Thus, sulfate reducing bacteria can cause harmful effects on the host intestinal barrier function in the setting of dysbiosis where SRB bloom is observed.

D. vulgaris increased tight junction permeability by causing upregulation and nuclear translocation of the nuclear transcription factor Snail with Snail responsible for downstream gene regulation that led to impairment of intestinal barrier function. Inhibiting Snail expression with siRNA reduced D. vulgaris-induced permeability. Snail expression increases under experimental conditions in response to various harmful stimuli. For example, LPS upregulates Snail and Snail induces barrier disruption and redistribution of tight junction proteins ZO-1 and occludin in response to acetaldehyde. In addition, overexpression of Snail induces disruption of tight junctions and induces permeability in MDCK canine kidney cells. Cells overexpressing Snail also showed a decreased protein expression of E-cadherin and claudin-1. Cells overexpressing Snail also show a redistribution of ZO-1 without affecting its protein expression. The redistribution of ZO-1 in the absence of change in protein expression is similar to our findings that D. vulgaris caused redistribution of occludin but did not affect its protein expression by Western blot.

### Role of Fusobacterium in Inducing Snail and Intestinal Permeability

Similar to Desulfovibrio, Fusobacterium is a hydrogen sulfide (H2S) producing gram-negative anaerobic opportunistic pathobiont that is typically found in low numbers in the gastrointestinal tract. However, overgrowth of Fusobacterium is observed in diseases such as inflammatory bowel disease and colorectal cancer where these bacteria may contribute to disease development. Further, Fusobacterium nucleatum, the most studied Fusobacterium species, shares common properties with Desulfovibrio in terms of its effect on mammalian cells in various experimental conditions. Exemplary similarities between the effects of F. nucleatum and Desulfovibrio on mammalian cells include, but are not limited to, increasing tight junction permeability; inducing the PI3K/Akt pathway, pro-inflammatory cytokines, and downstream signaling mediators; producing H2S as a byproduct of its metabolism; and increased bacterial density in the small intestine in antibiotic-treated subjects.

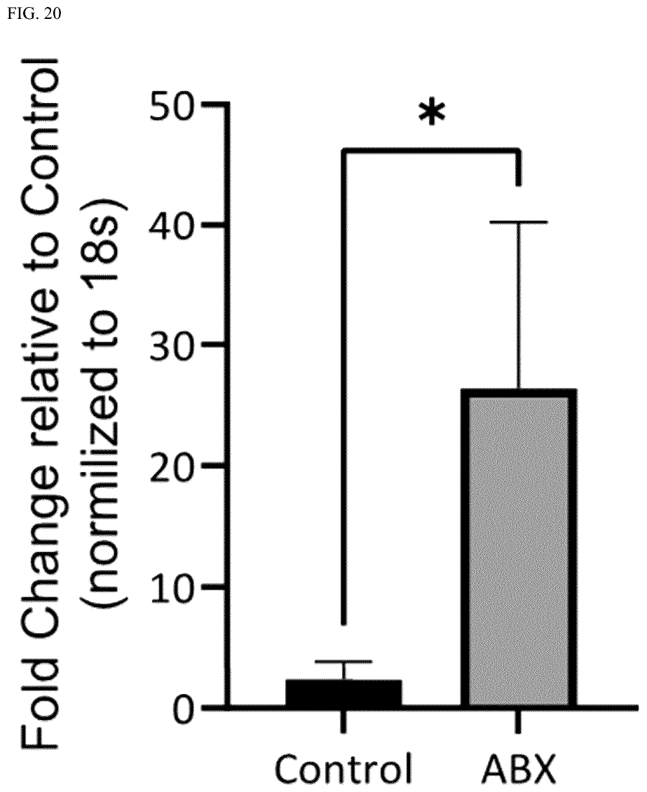

FIG. 20 shows that mice treated with an antibiotic cocktail, which induces gut microbial dysbiosis, exhibit a significant increase in the density of F. nucleatum in the small intestine, when compared to control untreated animals (Control 2.4±1.4 vs Antibiotic group (26.4±13.9, p<0.05). Desulfovibrio exhibit a similar increase in density in the small intestine in response to antibiotic treatment in mice.

### Methods of Treatment

In the description below, the term “SRB induced intestinal permeability” refers to impaired intestinal barrier function resulting from a bloom of hydrogen sulfide-producing bacteria. This disclosure further describes the use of a Snail inhibitor and/or an inhibitor of sulfate reducing bacteria to treat conditions. As used herein, “treat” or variations thereof refer to reducing, limiting progression, ameliorating, or resolving, to any extent, the symptoms or signs related to a condition. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject.

This disclosure describes the use of intestinal alkaline phosphatase as a model Snail inhibitor to treat sulfate reducing bacteria (SRB)-induced downregulation and/or mis-localization of tight junction proteins and adheren junction proteins. IAP reversed SRB-induced barrier dysfunction and inhibited SRB-induced increase in Snail protein expression and its nuclear translocation. IAP also corrected the mis-localization of occludin caused by D. vulgaris, an exemplary sulfate reducing bacterium. Exactly how IAP inhibits SRB-induced Snail remains unclear. Without wishing to be bound by any particular theory, it is possible that the effects of IAP are mediated through autophagy. For example, IAP induces autophagy in epithelial cells (Singh et al., 2020, Scientific Reports 10:3107), which helps maintain the barrier function and also targets Snail for degradation.

This disclosure therefore describes hydrogen sulfide producing bacteria as a contributor to the disease phenotype of leaky gut and identifies Snail expression as a novel therapeutic target for barrier permeability induced by hydrogen sulfide producing bacteria. This disclosure further identifies a model Snail inhibitor, intestinal alkaline phosphatase, as a therapeutic intervention for treating SRB-induced leaky gut by inhibiting expression of Snail. Thus, Snail inhibitors may be effective for treating any of the many inflammatory diseases that are linked to SRB overgrowth.

Snail signaling is regulated by ubiquitination and the ubiquitin proteasome degradation mechanism. The E3 ubiquitin ligases (F-box ligases) beta-TrCP1/Fbxw1, FBXL14, Fbxl5, FBX011, Fbxo45 and FBW7, the SOCS box protein SPSB3, and the HECT domain E3 ubiquitin ligase 1 (HECTD1) regulate the level of Snail by promoting its ubiquitination and degradation. These ligases are further regulated. For example, liver kinase B1 (LKB1) also known as serine-threonine kinase 11 (STK11) enhances interaction of E3 ligase FBXL14 with Snail, leading to greater ubiquitin-mediated degradation of Snail, an effect that is increased by metformin, a drug that could increase Snail ubiquitination by stimulating LKB1 expression. Snail signaling is also opposed by Notch signaling whereby Notch 1 intracellular domain (NICD) binds to the nuclear transcription factor Snail and induce its proteolytic degradation.

Working in the opposing direction are deubiquitinases such as DUB3, PSMD14, OTUB1, and USP36 that work to stabilize Snail through deubiquitination and reduced degradation. These deubiquitinases are also regulated. For example, CDK4/6 dephosphorylates DUB3 to enhance deubiquitination, leading to greater stability of Snail. This control point has provided a target for inhibiting Snail. Specifically, the drug palbociclib targets CDK4/6 to facilitate Snail ubiquitination and degradation by inhibiting phosphorylation of DUB3 by CDK4/6. Palbociclib also suppresses the expression of Snail. By inhibiting the deubiquitinase PSMD14, thiolutin facilitates greater ubiquitination and degradation of Snail, leading to Snail suppression. The small molecule CYD19 disrupts the interaction of Snail with CREB binding protein (CBP)/p300 to subsequently promote ubiquitination and degradation of Snail. Additional control that leads to increased Snail signaling include three survival kinases, ERK, AKT and IKKalpha/beta. AKT and ERK inhibit GSK3beta through its phosphorylation. Since GSK3β drives Snail nuclear exclusion via ubiquitination and degradation via beta-TrCP1, inhibition of GSK3beta would lead to greater availability of Snail. Additionally, ERK upregulates Snail transcription via the transcription factor AP-1. IKK-NF-κB pathway inhibits Snail by upregulating CSN2, an inhibitor of GSK3β. Exemplary Snail regulators, and exemplary activities, are summarized in Table 1. While exemplary activities are provided in Table 1, the regulators listed in Table 1 may target more than one pathway. thus, the listed activities should not be construed as being exhaustive.

TABLE 1
Snail regulators

| Decrease Snail | Increase Snail |
|---|---|
| F-box ligases- beta-TrCP1/Fbxw1, FBXL14, | Deubiquitinases- DUB3, PSMD14, OTUB1 |
| Fbxl5, FBX011, Fbxo45 and FBW7 | and USP36 |
| SOCS box protein SPSB3 | CDK4/6 |
| HECT domain E3 ubiquitin ligase 1 | Survival kinases (ERK, AKT and IKKα/β) |
| (HECTD1) | |
| LKB1/STK11 | IKK-NF-κB (by upregulating CSN2, an |
| | inhibitor of GSK3β) |
| Notch signaling | |
| Palbociclib (by targeting CDK4/6) | |
| Thiolutin (by inhibiting deubiquitinase | |
| PSMD14) | |
| CYD19 (by disrupting interaction of Snail | |
| with CBP/p300) | |
| GSK3β (drives ubiquitination and degradation | |
| via beta-TrCP1) | |
| Telocinobufagin (inhibits Snail in EMT in | |
| breast cancer cells) | |
| Toosendanin (inhibits TGF-β1-induced Snail | |
| in EMT) | |
| Ponicidin (inhibits TNF-induced Snail in | |
| EMT) | |
| Luteolin (inhibit TGF-β1-induced Snail in | |
| EMT via Pi3k/Akt.NF-κB inhibition) | |
| Berberine (Renal tubular EMT) | |
| Metformin (inhibits TGF-β1-induced Snail in | |
| EMT) | |
| Caffeine (inhibits Snail expression in cirrhotic | |
| rats) | |
| Neferine (inhibits Snail expression in EMT) | |
| Hispolon (inhibits TGF-βb1-induced Snail in | |
| EMT) | |
| Osthole (inhibits TGF-β1-induced Snail in | |
| EMT) | |
| Codonolactone (attenuates TGF-β signaling in | |
| EMT) | |
| Triptolide (attenuates TGF-β signaling in | |
| EMT) | |
| Galunisertib (LY2157299) (inhibits TGF-β | |
| signaling pathway in hepatocellular | |
| carcinoma cell line. | |
| LY364947 (inhibits both TGF-βR1 and TGF- | |
| βR2) | |
| SB431542 (specifically inhibits TGF-βR2) | |
| GW788388 (inhibits TGF-β type I and II | |
| receptor kinases in renal fibrosis) | |
| Resveratrol (inhibits TGF-β in LPS-induced | |
| EMT) | |
| Rapamycin (inhibits Snail in glioma cells) | |
| Torin (inhibits Snail in glioma cells) | |
| Emodin (inhibits Snail in autophagy- | |
| dependent manner) | |
| Trehalose (inhibits snail in autophagy | |
| dependent manner in Peritoneal fibrosis) | |

The methods described herein can alternatively be practiced using an inhibitor of sulfate reducing bacteria. Exemplary inhibitors of sulfate reducing bacteria include, but are not limited to, bismuth subsalicylate, molybdate, one or more antibiotics, and magnesium.

Multiple lines of evidence identify bismuth as an inhibitor of sulfate reducing bacteria. For example, bismuth inhibits the growth of Desulfovibrio desulfuricans. Further, sulfate reducing bacteria generate hydrogen sulfide (H₂S) as a product of their dissimilarity sulfate reduction pathway. Bismuth subsalicylate acts as a scavenger and binds H₂S, rendering it insoluble. Sulfate reducing bacteria slow down small intestinal transit by producing H₂S, which is reversed in the presence of bismuth subsalicylate. In addition, bismuth subsalicylate decreases the amount of H₂S measured from rat cecum and human stool by >95% via direct binding of this gas.

Sodium molybdate competitively inhibits the enzyme sulfate reduction pathways used by sulfate reducing bacteria and other HS-generating microbes in vitro. Inhibitory effects of molybdate on sulfate reducing bacteria have been demonstrated in environmental studies and in batch cultures.

A variety of antibiotics are active against hydrogen sulfide producing bacteria including but not limited to rifaximin, doxycycline, augmentin, and ciprofloxacin.

Magnesium, in the form of magnesium oxide (MgO) and MgO nanoparticles, possesses antimicrobial effects and is effective in suppressing the production of hydrogen sulfide. Further, in a batch culture of sulfate reducing bacteria, magnesium peroxide (MgO₂) inhibited the number of sulfate reducing bacteria, the sulfide concentration, and the sulfate reducing rate (SRR).

The methods described herein can alternatively be practiced using an inhibitor of TGF-β signaling and/or an inhibitor of PI3K/Akt signaling.

### DSV Induces Activation of PI3K/Akt/TNF/iNOS

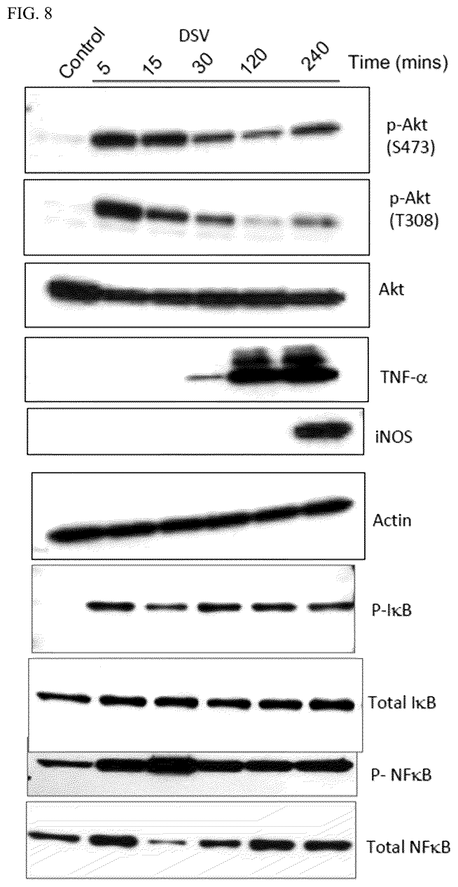

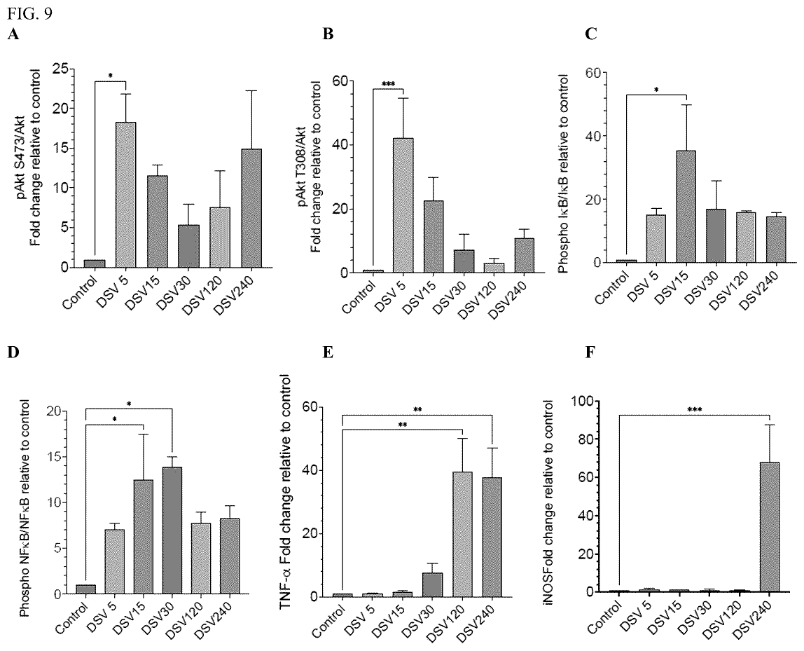

To test whether DSV induced PI3K/Akt/TNF and iNOS activation in a time-dependent manner, RAW cells were treated with DSV (MOI 20) for various time periods from five minutes to four hours. As early as five minutes after treatment with DSV, DSV induced phosphorylation of Akt at both Thr 308 and Ser 473 positions (FIG. 8) that are activated by PDK1 and PDK2, respectively. Values were significantly higher compared to control at five minutes post-infection for p-Akt S473 (DSV:18.33±3.45; Control:1.00, p<0.05, FIG. 9A) and p-Akt T308 (DSV: 42.35±12.30; Control:1.00, p<0.001, FIG. 9B). Levels of p-Akt dropped over time but the trend was consistently higher than control levels. Next, p-IκB and p-Nuclear factor-κB (NFκB) levels were measured since PI3K/Akt regulates phosphorylation of IκB and transcriptional activity of NF-κB. A significant increase in phospho-IκB levels occurred at 15 minutes post-infection when compared to Control (DSV: 35.41±14.38 vs Control: 1.00, p<0.05, FIG. 9C) as well as in phosphorylation of NF-κB p65 subunit at 15 minutes and 30 minutes (DSV15 mins: 12.47±4.97; DSV30 mins: 13.86±1.130 vs control:1.00, p<0.05, FIG. 9D). Induction of TNF-α occurred as early as 30 minutes and significantly peaked by two hours and remained high at four hours (DSV 2 hr: 39.51±10.47; DSV 4 hr: 37.92±9.01; Control: 1.00, p<0.01, FIG. 9E). On the other hand, induction of iNOS was observed only at four hours (DSV 4 h: 68.20±19.19; Control: 1.00, p<0.001, FIG. 9F). Thus, DSV induced components of PI3k/Akt pathway and downstream activation of TNF-α and iNOS in a time-dependent manner.

### Live and Heat-Killed DSV Induces PI3k/Akt/TNF/iNOS Pathway

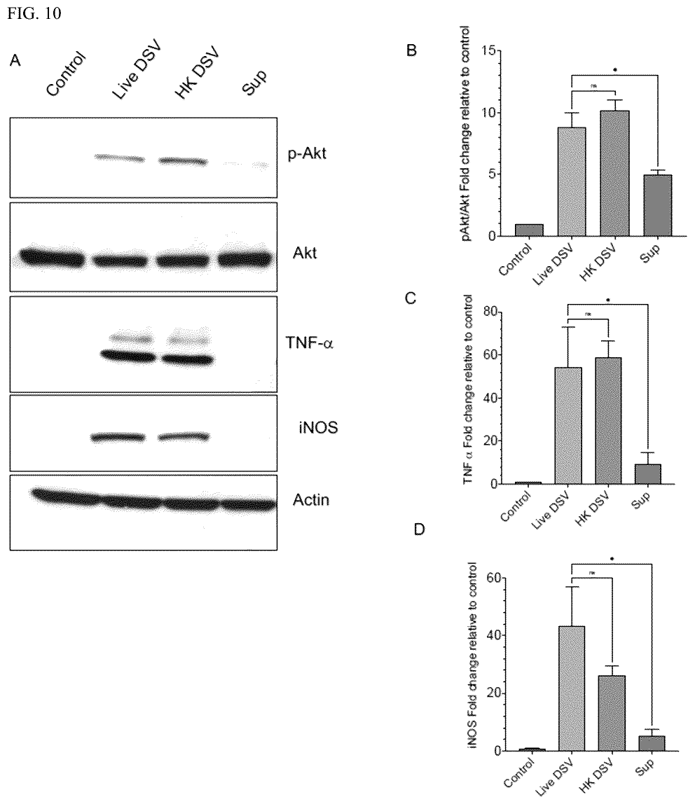

Next, the effects of live DSV, heat-killed DSV, and the culture supernatant of DSV on PI3K/Akt/TNF/iNOS pathway were compared (FIG. 10). For generating heat-killed bacteria (HK), DSV were autoclaved and equal volume of bacteria were added to the cells as live DSV. In the case of culture supernatant (Sup), 1 ml bacterial culture was centrifuged to pellet the bacteria. Sup was further passaged through a 0.2 μm filter to remove any remaining bacteria. Sixty microliters of supernatant, equivalent to the volume of resuspended bacteria at MOI 20, was applied to the cells. Heat-killed bacteria were equally efficient in inducing p-Akt compared to live bacteria (Live DSV: 8.79±1.15 vs HK: 10.12±0.91, p>0.05). However, levels of p-Akt were significantly lower in cells treated with Sup in comparison to live DSV (Live DSV: 8.79±1.15; Sup: 4.99±0.33; p<0.05, FIG. 10B). This trend was reflected in TNF-α expression where HK induced a comparable amount of the protein when compared to DSV but much lower induction of TNF-α by Sup (Live DSV: 54.37±18.62; HK: 59.10±7.28; p>0.05; Sup: 9.2915.41, p<0.01 compared to DSV, FIG. 10C). Similarly, HK bacteria induced iNOS levels comparable to DSV but in cells treated with culture sup, much lower levels of iNOS were induced (DSV: 22.37±3.44; HK: 20.16±3.54; p>0.05 compared to DSV; Sup: 5.51±2.62, p<0.01 compared to DSV, FIG. 10D). These results suggest that one or more structural components of DSV, rather than the metabolically active DSV, is responsible for inducing PI3K/Akt/TNF/iNOS pathway in macrophages. The structural component responsible for these effects of DSV may be an integral part of the bacteria or may be secreted in lower levels, based on our findings with milder effects of Sup when compared to live and HK bacteria.

Comparison of Effects of DSV and Lactobacillus plantarum on PI3k/Akt/TNF/iNOS Activation

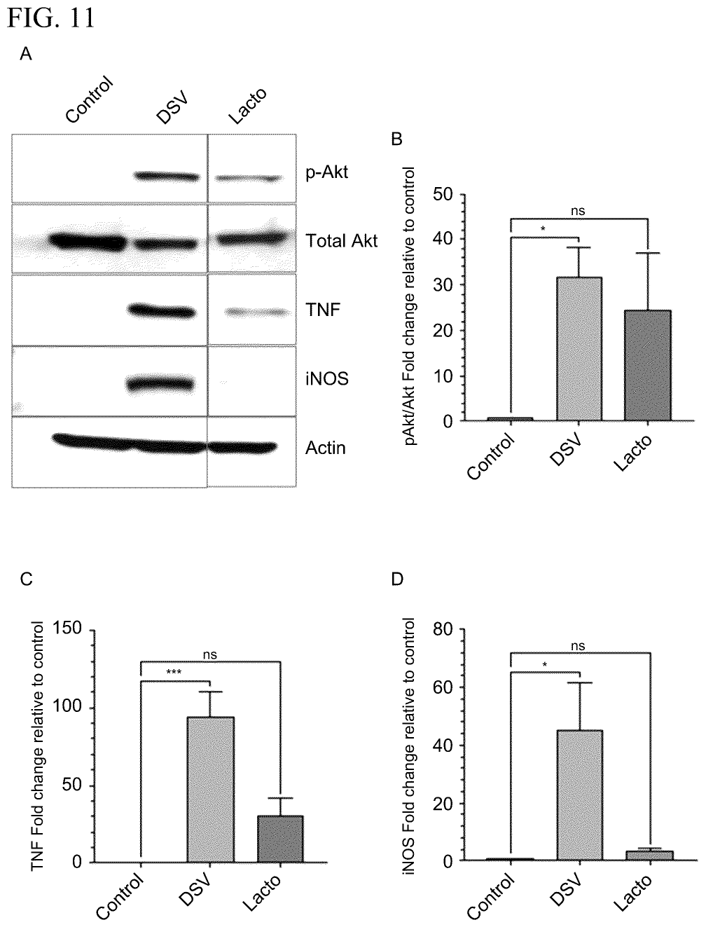

Next, whether a probiotic L. plantarum induced PI3K/Akt/TNF/NOS pathway was tested and the effects of L. plantarum were compared with those of DSV. Cells were treated with L. plantarum (Lacto) bacteria for four hours at MOI 20. DSV caused a significant increase in p-Akt when compared to Control (DSV: 31.75±6.54; Control:1.00,p<0.05). Interestingly, Lacto also induced p-Akt but the effects were milder than DSV and the levels were found to be statistically insignificant compared to Control (Lacto: 24.37±12.50; Control:1.00, p>0.05, FIG. 11 A,B). Also, Lacto caused an increase in TNF-α albeit to a much lesser extent than DSV (DSV: 101.5±20.63; Control 1.00, p<0.001) and these effects were also insignificant when compared to Control (Lacto: 22.65±9.187 vs Control: 1:00, p>0.05, FIG. 11A,C). Lacto failed to significantly induce iNOS expression (Lacto: 3.153±0.9706; Control:1.00, p>0.05, FIG. 11A,D), whereas DSV caused a drastic increase in iNOS compared to Control (DSV: 45.45±16.27; Control: 1.00, p<0.05). These findings suggest that these two bacteria diversify in eliciting cellular immune response downstream of PI3k/Akt and cells may generate different responses to a probiotic Lacto when compared to an opportunistic pathobiont DSV. These findings also support the notion that PI3K/Akt can be activated by both beneficial and harmful bacteria and the outcome may be determined by the bacteria and the host cells.

### LY294002 Inhibits DSV-Induced Activation of PI3K/Akt/TNF Pathway

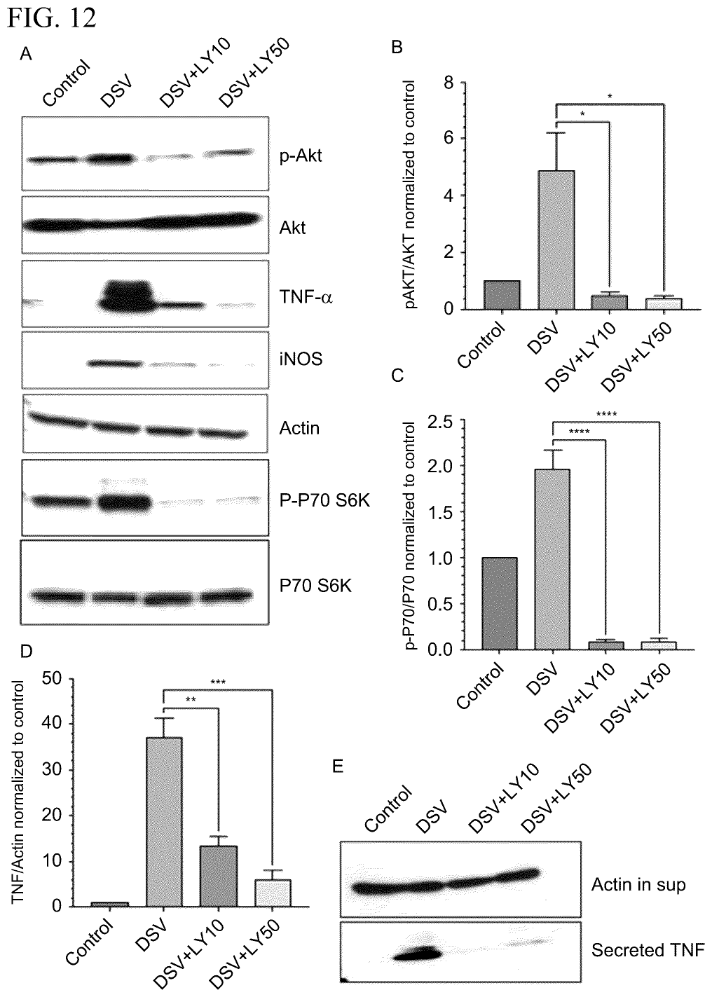

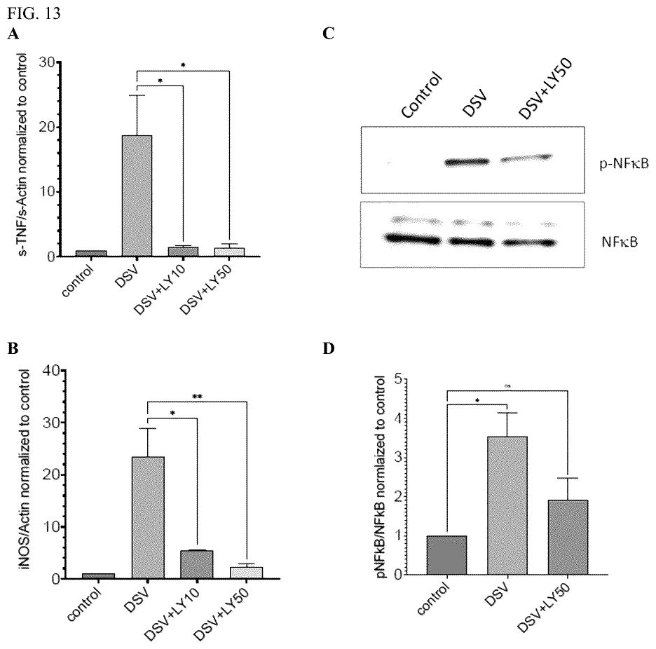

LY294002 (LY) is a well-known inhibitor of class I PI3K pathway. To investigate whether DSV induced classical PI3K/Akt pathway that was responsible for upregulating proinflammatory TNF-α and iNOS proteins, cells were pre-treated with LY for one hour before DSV challenge for four hours. LY inhibited DSV-induced Akt phosphorylation (DSV: 4.88±1.31; DSV+LY10: 0.52±0.11; DSV+LY50: 0.40±0.09, p<0.05 compared to DSV, FIG. 4A,B). P70S6K is another effector that is activated downstream of PI3K/Akt via phosphorylation by mTOR complex 1 (mTORC 1). LY inhibited DSV-induced p-P70 levels (DSV: 1.96±0.20; DSV+LY10: 0.081±0.026; DSV+LY50: 0.088±0.029, p<0.001 compared to DSV, FIG. 12A,C). Cellular TNF-α expression was elevated in the presence of DSV and LY significantly inhibited this effect (DSV: 37.01±4.28; DSV+LY10: 13.42±2.13, p<0.05; DSV+LY50: 6.044±2.139, p<0.01 compared to DSV, FIG. 12A,D). Similarly, levels of secreted TNF-α were inhibited by LY (DSV: 18.73±6.21; DSV+LY10: 1.45±0.24; DSV+LY50: 1.34±0.57, P<0.05 compared to DSV, FIG. 12E; FIG. 13A). LY also inhibited DSV-induced iNOS expression (DSV: 23.54±5.39; DSV+LY10: 5.45±0.17, p<0.05; DSV+LY50: 2.28±0.58, p<0.01, FIG. 13B). Next, expression of phospho-P65 subunit of NF-κB was measured to determine whether LY inhibited DSV-induced activation of NF-κB by measuring the (FIG. 13C,D). DSV treatment caused a significant increase in p-p65 when compared to Control (DSV: 3.54±0.59; Control: 1.00, p<0.05). However, prior treatment of cells with LY mitigated these effects of DSV (DSV+LY50: 1.91±0.54; Control: 1:00, p>0.05). These results suggest that DSV induced the classical PI3k/Akt pathway activation that was responsible for the downstream induction of proinflammatory TNF-α as well as iNOS.

### Activation of PI3/Akt Pathway by DSV is Dependent on TLR2 Signaling

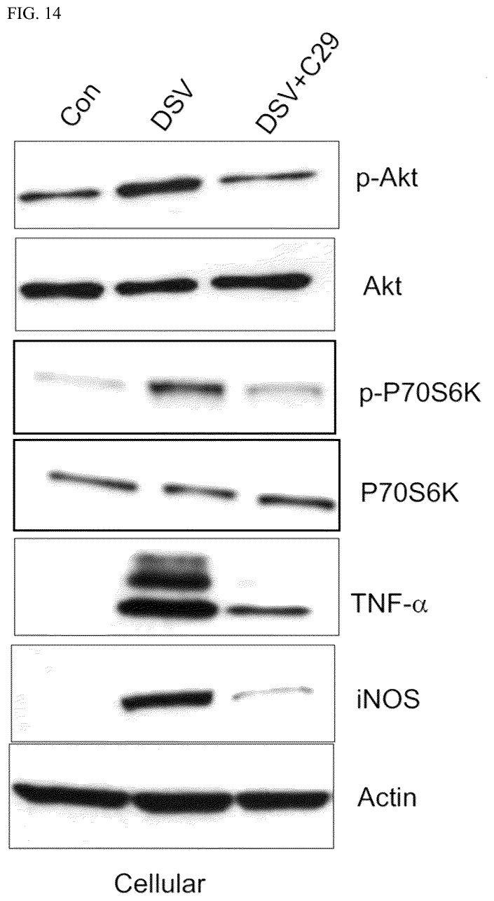

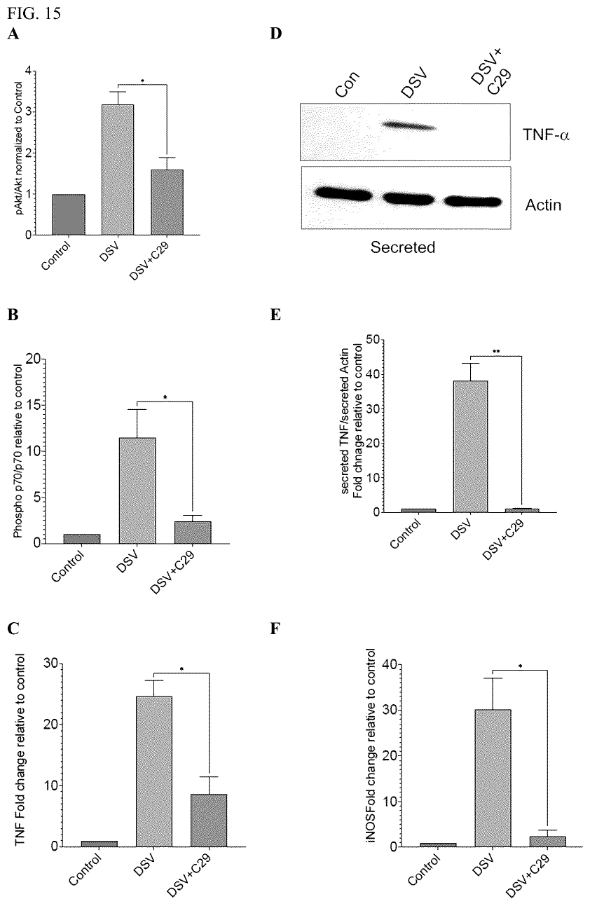

PI3k/Akt pathway is activated downstream of receptors such as RTKs and G-protein coupled receptors and also by TLRs activation which is often observed in the case of infections with various bacteria. Thus, cells were treated with different TLR antagonists to check for the involvement of TLR 2, TLR 4, or TLR 5 in DSV-mediated activation of PI3K/Akt pathway. Neither TLR4 antagonist TLR-IN-C34 nor TLR5 antagonist TH1020 had any discernable effect on DSV-induced PI3/Akt signaling. However, pretreatment of cells with TLR 2 inhibitor, TLR-C29 (C29) significantly inhibited DSV-induced effects (FIG. 14). DSV-induced phosphorylation of AKT was inhibited by C29 (DSV:3.18±0.30; DSV+C29: 1.58±0.29, p<0.05, FIG. 14, FIG. 15A). Similarly, expression of p-p70S6K, an effector downstream of Akt, was also upregulated by DSV and this was significantly inhibited by C29 (DSV: 11.48±3.05; DSV+C29: 2.3910.69, p<0.05, FIG. 14, FIG. 15B). Levels of cellular TNF-α protein were also upregulated by DSV and inhibited in the presence of C29 (DSV:24.67±2.51; DSV+C29: 8.57±2.81, p<0.05, FIG. 14, FIG. 15C). Also, levels of DSV-induced secreted TNF-α were significantly inhibited by C29 (DSV: 38.09±5.188; DSV+C29: 1.103±0.03408; control: 1.00 p<0.01, FIG. 15 D,E). Additionally, DSV-induced iNOS protein expression was significantly inhibited by C29 (DSV: 30.25±6.82; DSV+C29: 2.32±1.48, p<0.05, FIG. 14, FIG. 15F). PI3K/Akt is classically activated by a vast number of stimuli through receptor tyrosine kinases (RTK). Thus, tryphostin AS1478, a common tyrosine kinase inhibitor, was used as an antagonist to EGFR signaling to test whether DSV-induced PI3k/Akt/TNF/iNOS pathway was dependent on RTK-mediated signaling. No inhibition of the DSV-induced PI3K/Akt activation by EGFR antagonist was detected, suggesting that this RTK was not involved in DSV-mediated effects. Taken together, the results suggest that DSV specifically engages TLR 2 to mediate its pro-inflammatory effects via PI3K/Akt pathway.

Desulfovibrio are SRB that are present as minor members of the resident gut bacterial community and are mainly recognized for their production of hydrogen sulfide (H2S) as a byproduct of their metabolism. An increase in Desu/fovibrio density is observed in many diseases including IBD, metabolic syndrome, cancer, autism, and Parkinson's disease. Various cellular pathways that are affected by Desulfovibrio vilgaris include, but are not limited to, increased intestinal barrier permeability, decreased lysozyme production (a crucial anti-microbial protein), and increased pro-inflammatory Notch signaling pathway, effects that could contribute to disease development. This disclosure describes yet another layer of Desulfovibrio function that may be responsible for pathogenesis of diseases. Desulfovibrio vulgaris (DSV) caused an increase in protein expression of pro-inflammatory TNF-α and iNOS. While activation of immune response by bacteria is an important cellular defense mechanism to eliminate pathogens, uncontrolled activation of immune response is harmful and can lead to inflammation that underlies many acute and chronic diseases such as IBD and sepsis. It is possible that Desulfovibrio overgrowth, as seen in conditions such as IBD, may cause or promote the development of inflammatory diseases by activating a proinflammatory immune response. DSV-induced TNF-α and iNOS expression was dependent on PI3K/Akt pathway as inhibition of PI3K by LY294002 abrogated these effects. PI3K/Akt pathway is a signal transduction pathway that is involved in many fundamental cellular processes such as differentiation, survival, apoptosis, transcription, translation, proliferation, as well as in immune responses. PI3K/Akt cascade is activated by vast environmental stimuli via several cell surface receptors and orchestrates a plethora of downstream pathways via diverse effector molecules. Dysfunction of this pathway has been linked to many diseases such as metabolic diseases, cancer, neurodegenerative diseases, vascular diseases, infections, and inflammatory diseases. While it is involved in activating a pro-inflammatory response in some infections, in others it may play an anti-inflammatory role. Thus, the role of PI3K/Akt in infection and inflammation is multifaceted and diverse.

In this study, infection of RAW macrophages with L. plantarum induced PI3k/Akt expression, but mildly induced TNF-α and iNOS activation was negligible in the presence of these bacteria when compared to DSV. Similarly, Lactobacillus rhamnosus can induce M1 phenotype in macrophages to produce cytokines such as IL-1b, IL-10, and TNF-α in a TLR-2/MyD88/MAPK-dependent manner as a way to defend against pathogens. Thus, it is possible that probiotics may elicit a “healthy” immune response that can combat pathogens. On the other hand, the probiotic Lactobacillus paracasei induces M2 phenotype that is associated with an anti-inflammatory response. Thus, these overall disparate outcomes of PI3K/Akt activation may be dictated by the strain of the bacteria, their metabolic products, type of host cell, cell surface receptors and the overall disease context.

PI3K/Akt signaling has been vastly studied in the context of metabolism and differentiation downstream of growth factor receptors such as PDGF receptor (PDGFR) and epidermal growth factor receptor (EGFR), insulin-like growth factor receptor (IGFR) and insulin receptor (INSR), RAS, and G-protein coupled receptor, depending upon specific isoforms. However, PI3K/Akt pathway is also known to be activated in a toll-like receptor manner. TLRs other than TLR3 are known to activate NF-κB signaling and inflammatory cytokine production in a MyD88-dependent manner. The p85 subunit of PI3k binds to the cytosolic domain of TLR2 that contains PI3K binding motif (YXXM). TLRs activate PI3K/Akt pathway and further orchestrate downstream effects that are dictated by the type of stimulus. Engagement of TLR2, TLR4, and/or TLR5 with bacterial components leads to pro- or anti-inflammatory responses.

While inhibition of TLR4 and TLR 5 failed to inhibit DSV-induced PI3K/Akt/TNF/iNOS activation, significant inhibition of DSV-mediated activation of PI3k/Akt/TNF/iNOS pathway occurs in the presence of C29, a potent antagonist of TLR2/1 and TLR2/6 signaling that has been shown to prevent TNF-α production by bacteriocins. While TLR 2 is mostly known to be activated in response to gram-positive bacteria, TLR 2 activation can occur in response to gram-negative bacteria such as Porphyromonas gingivalis, Treponema pallidum, and Vibrio cholerae. Which component of DSV induces TLR2 signaling remains to be determined. Decreased levels of HS and Desulfovibrio spp. in the colon/feces occurs in TLR 2^{-/-} mice, while levels of H2S increased in TLR4^{-/-} mice, suggesting a link between SRB and TLR2 in vivo. Moreover, higher expression of TLR2 has been found in IBD patients. Additionally, higher levels of fecal H₂S are observed in IBD patients. These studies suggest a link between SRB, H₂S and TLR2 signaling.

Induction of TNF-α and iNOS by DSV is physiologically relevant as high levels of these proinflammatory mediators and their downstream products such as reactive nitrogen species are seen in inflammatory diseases such as IBD. In fact, anti-TNF-α antibodies are considered as therapeutic avenues for managing IBD since TNF is an inflammatory mediator in these diseases. TNF-α can further activate NF-κB and proinflammatory PI3K/Akt pathway. Thus, production and secretion of TNF-α by DSV-infected cells can initiate a PI3K/Akt cascade in uninfected cells further amplifying the inflammatory events.

The discovery of DSV-induced iNOS expression supports previous findings where DSV induced nitrite production in RAW cells. The role of iNOS in inflammation is dichotomous, being both harmful and beneficial to the host. iNOS activation in immune cells occurs in response to infections, leading to increased production of nitric oxide (NO) that is responsible for killing and limiting the infectious agents. However, accumulation of iNOS and NO as seen in IBD may have opposite effects, leading to chronic inflammation in these diseases. As iNOS is capable of producing NO over days, in the setting of Desulfovibrio bloom in inflammatory conditions with dysfunctional immune response, this may lead to chronic accumulation of NO production which can further exacerbate inflammation. Moreover, TNF-α treatment itself can lead to increased production of reactive nitrogen intermediates. Thus, results described herein suggest that production of TNF-α and iNOS in response to DSV may be responsible, at least in part, for inflammation in diseases such as IBD where Desulfovibrio overgrow. Taken together, the data provided herein identify a novel pathway by which SRB such a Desulfovibrio may induce inflammatory events and may explain the mechanisms underlying the association between a DSV bloom and inflammatory diseases. The data therefore further identify novel targets for therapeutic strategies to treat diseases associated with DSV overgrowth.

Inhibitors of Pak1 and activators of GSK3β regulate Snail nuclear localization via its phosphorylation. Snail is a highly labile protein; its function is tightly regulated by its phosphorylation status, which is mediated by various regulators. Among these, Pak1 mediates snail phosphorylation at Ser 246, which causes its nuclear transport and stabilization, which is important for Snail function. In contrast, phosphorylation of snail by GSK3p at various serine positions (e.g., S¹⁰⁷, S¹¹¹, S¹¹⁵, S¹¹⁹), causes its nuclear export and degradation by proteasomes, thus inhibiting snail function.

Given the role of SRB in inducing mRNA and protein expression of Snail and/or nuclear translocation, SRB may cause an increase in PAK1 expression and activity, which results in Snail activation. Conversely, SRB may inhibit GSK3β activity, thus enabling Snail function. Thus, inhibitors of PAK1 and activators of GSK3β are putative interventions to inhibit SRB-induced Snail activation and function.

Exemplary Snail regulators, and exemplary activities, are summarized in Tables 2-6. While exemplary activities are provided in Tables 2-6, the regulators listed in Tables 2-6 may target more than one pathway. Thus, the listed activities should not be construed as being exhaustive. Further, Snail inducing and Snail inhibitory described herein also include all the downstream mediators of each of these pathways, that may directly or indirectly regulate SRB-induced Snail expression and barrier permeability. Such mediators include, but are not limited to, mTOR, STAT3, Smad, ERK, JNK, MAPK, cytokines, reactive oxygen species (ROS), and MLCK that are activated downstream of the abovementioned regulators.

TABLE 2
TLR2/PI3K/Akt inhibitors

| Inhibitor | Action |
|---|---|
| Simvastatin | Inhibits Pi3K/Akt in EMT in esophageal |
| | cancer cells |
| α-mangostin | Inhibits EMT by downregulating PI3K/Akt |
| | pathway |
| Curcumin | Superoxide dismutase-induced epithelial-to- |
| | mesenchymal transition via the PI3K/Akt/NF- |
| | κB pathway |
| Isoviolanthin | Inhibits TGF-β1-Mediated EMT by |
| | deactivating the TGF-β/Smad and |
| | PI3K/Akt/mTOR signaling pathways |
| Trichostatin A | Suppresses Akt pathway in mechanical |
| | ventilation-augmented bleomycin-induced |
| | EMT |
| LY294002 | Inhibits PI3K/Akt pathway |
| C29 | Inhibits TLR2 signaling |
| Phloretin | Inhibits TLR2/NFκB signaling |
| Epigallocatechin-3-gallate (EGCG) | Inhibits TLR2 |
| Naringenin | Inhibits TLR2 |
| Telocinobufagin | Inhibits Snail in EMT in breast cancer cells |

TABLE 3

| Autophagy Inducers | PI3K and Akt Inhibitors |
|---|---|
| Carbamazepine (PMID: 20522742) | Perifosine (PMID: 19920197) |
| Curcumin (PMID: 26573768) | Quercetin (PMID: 21610320) |
| Salvianolic acid B (PMID: 27557491) | NVP-BEZ235 & PI-103 (PMID: 21062993) |
| Tvrosine kinase inhibitors (PMID: 23737459) | GDC-0980 & Gedatolisib (PMID: 30782187) |
| Resveratrol(PMID: 21364612) | NVP-BKM120 (PMID: 22188813) |
| Spermidine (PMID: 19801973) | GDC-0068 (PMID: 23287563) |
| Apigenin (PMID: 26292725) | MK-2206 (PMID: 22025163) |
| | Afuresertib (PMID: 25075128) |

TABLE 4

| NFκB Inhibitors | TNF α Inhibitors |
|---|---|
| BAY-11-7082 (PMID: 29771406) | Infliximab, Adalimumab, |
| Losartan (PMID: 11862111) | Etanercept, Golimumab, and |
| Thiopental (PMID: 11981162) | Certrolizumab |
| Cyclosporin A (PMID: 9247567) | (PMID: 25624856) |
| Glucosamine sulphate (PMID: 12681956) | |
| Genistein (PMID: 9521814) | |
| Hydroxyquinone (PMID: 12699902) | |
| Aspirin (Sodium Salicylate) | |
| (PMID: 8533099) | |

TABLE 5
TGFβ inhibitors

Fresolimumab (PMID: 24618589)
LY3022859 (PMID: 20145179)
P144 (PMID: 27473823)
Vactosertib (PMID: 31673825)
Galunisertib (PMID: 35738451)
LY3200882 (PMID: 35462305)
Trabedersen (PMID: 19579166)

TABLE 6

| Pak1 inhibitors | GSK3β Activators |
|---|---|
| FRAX486 PMID: 23509247 | Secalonic acid D |
| FRAX597 (PMID: 23960073) | PMID: 19571678 |
| G-5555 (PMID: 26713112) | DIF-3 (PMID: 25913757) |
| PF-3758309 (PMID: 20439741) | |
| IPA-3 (PMID: 19723886) | |
| NVS-PAK1-1 (Compound 3) | |
| PMID: 26191365 | |

Treating a condition can be prophylactic or, alternatively, can be initiated after the subject exhibits one or more symptoms or clinical signs of the condition. Treatment that is prophylactic—e.g., initiated before a subject manifests a symptom or clinical sign of the condition such as, for example, while overgrowth of sulfate reducing bacteria is subclinical—is referred to herein as treatment of a subject that is “at risk” of having the condition. As used herein, the term “at risk” refers to a subject that may or may not actually possess the described risk. Thus, for example, a subject “at risk” of a condition involving gut permeability is a subject possessing one or more risk factors associated with conditions involving gut permeability such as, for example, genetic predisposition, ancestry, age, sex, geographical location, lifestyle, or medical history. Treatment may also be continued after symptoms have resolved, for example to prevent or delay their recurrence.

Accordingly, a composition can be administered before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering an effective amount of a therapeutic composition that includes a Snail inhibitor or an inhibitor of sulfate reducing bacteria (collectively hereafter, “active agent”) to a subject having, or at risk of having, a particular condition. In this aspect, an “effective amount” is an amount effective to reduce, limit progression, ameliorate, or resolve, to any extent, a symptom or clinical sign related to the condition. For example, an effective amount can be an amount of the active agent effective to inhibit overgrowth or bloom of sulfate reducing bacteria. In other embodiments, an effective amount can be an amount effective to decrease Snail expression in treated cells compared to untreated cells, decrease FITC-dextran flux in treated cells compared to untreated cells, decrease the percentage of cells with Snail localized in the nucleus in treated cells compared to untreated cells, decrease cytoplasmic localization of occludin in treated cells compared to untreated cells, and/or increase paracellular localization of occluding in treated cells compared to untreated cells.

The composition containing one or more active agents, as described herein, may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with intestinal alkaline phosphatase without causing any undesirable biological effects or interacting in a deleterious manner with any of the other components of the pharmaceutical composition in which it is contained.

The composition containing one or more active agents may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, the pharmaceutical composition containing one or more active agents may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the active agent(s) into association with a carrier that constitutes one or more accessory ingredients. In general, a formulation may be prepared by uniformly and/or intimately bringing the active agent(s) into association with a liquid carrier, a finely divided solid carrier, or both, and then, if necessary, shaping the product into the desired formulations.

The amount of active agent(s) administered can depend on various factors including, but not limited to, the weight, physical condition, and/or age of the subject, and/or the route of administration. Thus, the absolute amount of active agent(s) included in a given unit dosage form can vary widely, and depends upon factors such as the species, age, weight, and physical condition of the subject, and/or the method of administration. Accordingly, it is not practical to set forth generally the amount that constitutes an amount of active agent(s) effective for all possible applications. Those of ordinary skill in the art, however, can readily determine the appropriate amount with due consideration of such factors.

For example, an active agent may be administered at the same dose and frequency for which it has received regulatory approval and/or, in the absence of needing regulatory approval, is customarily used. In other cases, an active agent may be administered at the same dose and frequency at which it may be being evaluated in clinical or preclinical studies. One can alter the dosages and/or frequency as needed to achieve a desired level of therapeutic or prophylactic affect. Thus, one can use standard/known dosing regimens and/or customize dosing as needed.

In one or more embodiments, the method can include administering sufficient intestinal alkaline phosphatase to provide a dose of, for example, from about 200 U/kg to about 1000 U/kg to the subject, although in one or more embodiments the methods may be performed by administering intestinal alkaline phosphatase in a dose outside this range.

A single dose may be administered all at once, in multiple discrete administrations, or continuously for a prescribed period of time. For example, one dosing regimen for the model Snail inhibitor intestinal alkaline phosphatase includes administering 67.5 U/kg over 10 minutes via intravenous bolus, followed by administering 132.5 U/kg via intravenous infusion over up to 48 hours.

When multiple administrations are used, the amount of each administration may be the same or different. For example, a dose of 1000 U/kg/day may be administered as a single administration of 1000 U/kg, continuously over 24 hours, as two or more equal administrations (e.g., two 500 U/kg administrations), or as two or more unequal administrations (e.g., a first administration of 750 U/kg mg followed by a second administration of 250 U/kg). When multiple administrations are used to deliver a single dose, the interval between administrations may be the same or different.

In one or more embodiments, the active agent may be administered, for example, from a single dose to multiple doses per week, although in one or more embodiments the method can involve a course of treatment that includes administering doses of the active agent at a frequency outside this range. When a course of treatment involves administering multiple doses within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

Conditions associated with SRB-induced leaky gut that may be treated as described above include, but are not limited to, gastrointestinal conditions, autoimmune conditions, extra-intestinal conditions, and neurobehavioral conditions.

Exemplary gastrointestinal conditions include, but are not limited to, inflammatory bowel disease, celiac disease, autoimmune hepatitis, alcoholic liver disease, nonalcoholic liver disease, cirrhosis, food allergy, food hypersensitivity, eosinophilic esophagitis, sclerosing cholangitis, pancreatitis, irritable bowel syndrome, and spontaneous bacterial peritonitis.

Exemplary autoimmune conditions include, but are not limited to, asthma, type 1 diabetes, type 2 diabetes, ankylosing spondylitis, rheumatoid arthritis, systemic lupus erythematosus, and allergic rhinitis.

Exemplary extra-intestinal conditions include, but are not limited to, fibromyalgia, chronic fatigue syndrome, obesity, cancer, polycystic ovary syndrome, eczema, psoriasis, acne, atopic dermatitis, cardiovascular disorders, chronic kidney diseases, metabolic syndrome, intestinal graft vs. host disease, human immunodeficiency syndrome, aging, multiorgan failure syndrome, endurance exercises, burns and pregnancy, obstructive sleep apnea, systemic hypertension, and musculoskeletal injuries.

Exemplary neurobehavioral conditions include, but are not limited to, multiple sclerosis, Parkinson's disease, Alzheimer disease, Lewy Body Dementias, schizophrenia, depression, hepatic encephalopathy neurodegenerative diseases, mild cognitive impairment, autism spectrum disorders, attention deficit hyperactivity disorder (ADHD), migraines, amyotrophic lateral sclerosis, dipolar disease, and anxiety.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” or “one or more embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Cell Culture and Treatments

Human colonic epithelial cells Caco-2 were purchased from the American Type Culture Collection (ATCC, Manassas, VA). Cells were grown in DMEM+20% FBS+penicillin and streptomycin (Thermo Fisher Scientific, Inc., Waltham, MA). Cells were grown at 37° C. in a humidified incubator with 5% CO₂. To allow polarization and differentiation, cells were seeded at a density of 5×10³ cells/well in 12-well 0.4 μm trans-well inserts for three weeks. Twenty-four hours before infection, growth medium was replaced with colorless medium (DMEM+20⁰/o FBS) without antibiotics. Cells were treated with Desulfovibrio vulgaris at various multiplicity of infection (MOI) for 24 hours. For intestinal alkaline phosphatase (IAP) treatment, cells were incubated with 500 U/ml of IAP (Sigma-Aldrich, St. Louis, MO) or vehicle alone (solution in which IAP was supplied) at the apical and basolateral surface for 24 hours prior to D. vulgaris challenge.

Desulfovibrio vulgaris (DSV) Growth

Desulfovibrio vulgaris Hildenborough (ATCC 29579, Manassas, VA) was grown anaerobically in Hungate tubes using Postgate's organic liquid medium. Media composition: 10.56 mM Na₂SO₄, 13.29 mM MgSO₄, 4.12 mM L-Cysteine, 0.4% sodium lactate (60% syrup), 0.4% yeast extract, and 0.5% tryptone. Cultures were grown for ˜24 hours in 5-ml aliquots at 37° C. Bacteria were counted using Quantom Tx cell counter (Logos Biosystems, Anyang, South Korea) and a Petroff Hausser counting chamber (Hausser Scientific Co., Horsham, PA). Before infection, bacteria were pelleted (6000 rpm for five minutes) and resuspended in phosphate buffered saline solution (PBS). For heat-killed bacteria (HK), D. vulgaris was autoclaved and the volume equivalent to the original count was used for infections. For obtaining bacterial culture supernatant, live D. vulgaris were centrifuged and the supernatant was passaged through a 0.2-μm filter to remove bacteria. 100 μl of this supernatant was added to Caco-2. As a control for this, 100 μl of Postgate's medium was added to the cells. Cells were infected with live bacteria, heat-killed bacteria, or supernatant for 24 hours.

### TEER and FITC Flux

Tight junction barrier integrity in 21-day old polarized Caco-2 cells grown in 12-well trans-well plates was assessed via transepithelial electric resistance (TEER) using EVOM meter (World Precision Instruments, LLC, Sarasota, FL). Cells with TEER >450 S were used in the experiments. Paracellular permeability was quantified by measuring FITC-dextran flux (Fluorescein isothiocyanate-dextran 4000 (FD-4), Sigma-Aldrich, St. Louis, MO). A solution of 25 mg/ml of FD-4 was made in sterile PBS. 40 μl of this solution was added apically to the cells 24 hours post-infection with D. vulgaris. After one hour of incubation, 100 μl of the basolateral medium was collected and analyzed for the presence of FITC fluorescence. Fluorescence was measured at the wavelengths of excitation at 485 nm and emission at 520 nm using a plate reader (Synergy HTX Multi-Mode Reader, BioTek, Winooski, VT). All the tests were carried out in triplicate.

siRNA Transfection

Cells were transfected with either control scrambled (Scr) or with Snail silencer select siRNA (Thermo Fisher Scientific, Inc., Waltham, MA) (35 nM) with lipofectamine RNAiMAX (Thermo Fisher Scientific, Waltham, MA) using manufacturer's instructions. Briefly, cells (1×10⁵/well) were seeded into a 12-well trans-well plate and grown for 21 days. siRNAs mixed with lipofectamine RNAiMAX in OptiMEM medium were added to the apical surface of the trans-well for 48 hours. First, transfection efficiency was measured by using two different siRNAs against Snail, either alone or in combination. Transfection efficiency was measured by Western blotting to assess the levels of Snail protein. After 48 hours, cells were infected with D. vulgaris (MOI20) for 24 hours. Permeability was assessed by measuring FITC-Dextran flux.

Cells were harvested and analyzed for Snail expression by Western blot.

### Western Blot

Cells were lysed in lysis buffer (Thermo Fisher Scientific, Inc., Waltham, MA; catalog #87787) containing protease and phosphatase inhibitors (Thermo Fisher Scientific, Inc., Waltham, MA; catalog #1861281) for 20 minutes at 4° C. with shaking. Lysates were collected and centrifuged at 12000 rpm for five minutes at 4° C. Protein concentration in the supernatants was determined with Bradford reagent (Bio-Rad Laboratories, Inc., Hercules, CA). 50 μg of protein samples were run on SDS-PAGE (4-20% tris-glycine) and transferred to nitrocellulose membranes. Membranes were blocked in 5% milk in PBS-T (0.1% Tween 20) for 30 minutes followed by overnight incubation in antibodies against actin (Cell Signaling Technology, Danvers, MA) and Snail (Cell Signaling Technology, Danvers, MA) Antibodies were diluted as recommended by the manufacturer. Blots were incubated with secondary antibodies (Cell Signaling Technology: #7074) at room temperature for one hour (dilution of 1:2000) and developed using enhanced Chemiluminescence HRP signal (Thermo Fisher Scientific, Inc., Waltham, MA).

### Immunofluorescence

Polarized Caco-2 cells were fixed with 4% paraformaldehyde for 15 minutes. Trans-wells were then washed apically and basolaterally with PBS three times, for five mins per wash. Cells were blocked in a blocking solution consisting of 5% FBS and 0.3% Triton-X 100 for one hour. This was followed by incubation of both apical and basolateral surface of the trans-wells with primary antibody against Snail1 or occludin (Cell Signaling Technology, Danvers, MA) at 4° C. Cells were then washed with PBS followed by incubation with secondary anti-rabbit antibody (Thermo Fisher Scientific, Inc., Waltham MA; catalog #A21206) for two hours at room temperature. Imaging was done with confocal microscope (Olympus Corp., Tokyo, Japan) using Z-stacks. Final images were presented as compressed Z-stack images to represent the entire depth of the monolayer for an unbiased view of the tight junctions.

### Statistical Analysis

All graphs were generated using PRISM 8 software (GraphPad Software, San Diego, CA). Data represents Mean±SEM from at least three independent experiments. Values were normalized to control and presented as percent change relative to Control set at 100%. Three or more groups were compared with one-way ANOVA. Two groups were compared with students t-test was used for statistical analysis. P values <0.05 were considered statistically significant.

### Example 2

Cell Culture and Treatments

RAW 264.7 murine macrophage-like cells were grown in DMEM+10% FBS without any use of antibiotics or antimycotics. Cells were grown at 37° C. in a humidified incubator with 5% CO₂. Prior to the day of infection, 8×10⁵ cells were plated in a six-well plate and infected with DSV at multiplicity of infection 20 (MOI 20) for various times. For drug treatments, cells were incubated with LY294002 at 10 μM or 50 μM for one hour followed by DSV infection for 30 minutes or four hours. For TL2-C29 treatment, cells were incubated with the inhibitor at 200 μM for three hours followed by infection with DSV for four hours.

### Bacteria

Desulfovibrio vulgaris Hildenborough (ATCC 29579) was grown anaerobically for 24 hours in Hungate tubes containing Postgate's organic liquid medium. Media composition: 10.56 mM Na₂SO₄, 13.29 mM MgSO₄, 4.12 mM L-Cysteine, 0.4% sodium lactate (60% syrup), 0.4% yeast extract, and 0.5% tryptone. Cultures were grown for approximately 24 hours in 5 ml aliquots at 37° C. Lactobacillus plantarum (ATCC 8014) were grown in Lactobacilli MRS Broth (BD288130) in a 37° C. incubator for 24 hours in 10 ml medium. Bacteria were counted using a cell counter (QUANTOM Tx, Logos Biosystems, South Korea).

### Western Blotting

Cells were lysed in Lysis buffer containing protease and phosphatase inhibitors (Thermo Fisher Scientific, Inc., Waltham, MA) for 20 minutes at 4° C. Lysates were centrifuged at 12,000 rpm for five mins at 4° C. and supernatants were collected. Protein concentration in the supernatants was determined with Bradford reagent (Bio-Rad Laboratories, Inc., Hercules, CA). 50 μg of protein samples were run on SDS-PAGE (4-20% tris-glycine) and transferred to nitrocellulose membranes. Membranes were blocked in 5% milk in PBS-T (0.1% Tween 20) for 30 minutes followed by overnight incubation in primary antibodies in cold room. All antibodies except iNOS were purchased from Cell Signaling Technology (Danvers, MA): Actin (4970), p-AktS473(4060), p-Akt T308(13038), Akt (9272), p-P70S6K (9205), P70S6K (2708, TNF-α (19948). iNOS antibody was purchased from Abcam (Cambridge United Kingdom). Antibodies were diluted as recommended by the manufacturer. The following day, blots were washed with PBS-T and incubated with secondary antibodies (Cell Signaling Technology, Danvers, MA) at room temperature for one hour (dilution of 1:2000) and developed using enhanced chemiluminescence HRP signal (Thermo Fisher Scientific, Inc., Waltham, MA).

### Statistical Analysis

All graphs were generated using PRISM 9.4.1 GraphPad Software Inc., San Diego, CA). Data is plotted as Mean±SEM relative to control. Each experiment was conducted at least three independent times. For western blot analysis, within each independent experiment, three biological replicates were used and combined to represent one treatment group. For data analysis, comparison between two groups was made using a two-tailed t-test. One-way ANOVA with a post-hoc Dunnett's multiple comparison test was used for comparing the difference between three or more groups. P values <0.05 were considered significant.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method of treating a subject having, or at risk of having, leaky gut caused by hydrogen sulfide producing bacteria, the method comprising:
administering to the subject an amount of a composition effective to reduce ameliorate at least one symptom or clinical sign of leaky gut caused by hydrogen sulfide producing bacteria, the composition comprising a Snail inhibitor or an inhibitor of sulfate reducing bacteria.

2. The method of claim 1, wherein the composition is administered in an amount effective to inhibit overgrowth or bloom of hydrogen sulfide producing bacteria.

3. The method of claim 1, wherein the composition is administered in an amount effective to decrease Snail expression in treated cells compared to untreated cells.

4. The method of claim 1, wherein the composition is administered in an amount effective to decrease FITC-dextran flux in treated cells compared to untreated cells.

5. The method of claim 1, wherein the composition is administered in an amount effective to decrease the percentage of cells with Snail localized in the nucleus in treated cells compared to untreated cells.

6. The method of claim 1, wherein the composition is administered in an amount effective to decrease cytoplasmic localization of occludin in treated cells compared to untreated cells.

7. The method of claim 1, wherein the composition is administered in an amount effective to increase paracellular localization of occluding in treated cells compared to untreated cells.

8. The method of claim 1, wherein the Snail inhibitor comprises an inhibitor of TLR2 signaling, an inhibitor of PI3K/Akt signaling, an inhibitor of TGF-β signaling, an inhibitor of TNF-α, an inhibitor of NF-κB, an inhibitor of Pak1, an inducer of GSK3β, or an inducer of autophagy.

9. The method of claim 1, wherein the inhibitor of sulfate reducing bacteria comprises bismuth subsalicylate, molybdate, or magnesium.

10. The method of claim 1, wherein the hydrogen sulfide producing bacteria comprise sulfate reducing bacteria.

11. A method of inhibiting deterioration of cellular junctions in cells contacted with hydrogen sulfide producing bacteria, the method comprising:
contacting the cells with an amount a composition effective to reduce the extent to which the hydrogen sulfide producing bacteria deteriorate cellular junctions compared to untreated cells, the composition comprising a Snail inhibitor or an inhibitor of hydrogen sulfide producing bacteria.

12. The method of claim 11, wherein the amount of composition is an amount effective to decrease Snail expression in treated cells compared to untreated cells.

13. The method of claim 11, wherein the amount of the composition is an amount effective to decrease FITC-dextran flux in treated cells compared to untreated cells.

14. The method of claim 11, wherein the amount of the composition is an amount effective to decrease the percentage of cells with Snail localized in the nucleus in treated cells compared to untreated cells.

15. The method of claim 11, wherein the amount of the composition is an amount effective to decrease cytoplasmic localization of occludin in treated cells compared to untreated cells.

16. The method of claim 11, wherein the amount of the composition is an amount effective to increase paracellular localization of occluding in treated cells compared to untreated cells.

17. The method of claim 11, wherein the Snail inhibitor comprises an inhibitor of TLR2 signaling, an inhibitor of PI3K/Akt signaling, an inhibitor of TGF-β signaling, an inhibitor of TNF-α, an inhibitor of NF-κB, an inhibitor of Pak1, an inducer of GSK3β, or an inducer of autophagy.

18. The method of claim 11, wherein the inhibitor of hydrogen sulfide producing bacteria comprises bismuth subsalicylate, molybdate, one or more antibiotics, or magnesium.

19. The method of claim 10, wherein the hydrogen sulfide producing bacteria comprise sulfate reducing bacteria.
